SEQUENCE LISTING (2 sequences)

SEQ ID NO: 1 - protein, 10 aa, Artificial sequence
  Synthetic sequence
HAIMGVAFTW

SEQ ID NO: 2 - protein, 22 aa, Artificial sequence
  Synthetic sequence
HAIMGVAFTWVMALACAAPPLV

(19) United States
(12) Patent Application Publication
(10) Pub. No.: US 2010/0015120 A1
(43) Pub. Date: Jan. 21, 2010

(54) HYBRID PROTEIN THAT CONVERTS ARACHIDONIC ACID INTO PROSTACYCLIN

(76) Inventor: Ke-He Ruan, Bellaire, TX (US)

Correspondence Address: CONLEY ROSE, P.C.;David A. Rose, P. O. BOX 3267, HOUSTON, TX, 77253-3267, US

(21) Appl. No.: 12/282,085

(22) PCT Filed: Mar. 8, 2007
(86) PCT No.: PCT/US07/63542

§ 371 Date: May 18, 2009

Related U.S. Application Data
(60) Provisional application No. 60/780,120, filed on Mar. 8, 2006.

(51) Int. Cl. A61K 38/44; C12N 9/02; C07H 21/04; C12N 15/63; C12N 5/00; C12P 21/06; A61K 31/7088

(52) U.S. Cl. 424/94.4; 435/189; 536/23.2; 435/320.1; 435/325; 435/69.1; 514/44 R

(57) ABSTRACT

A recombinant 130-kDa protein is constructed by linking together human cyclooxygenase (COX) isoform-2 (COX-2) and prostacyclin synthase (PGIS), via a 10-20 amino acid residues of a transmembrane sequence. The engineered protein is expressed in cells, and adopts the functions of COX and PGIS, to continually convert arachidonic acid (AA) into prostaglandin G₂ (catalytic step 1), prostaglandin H₂ (catalytic step 2) and prostacyclin (PGI₂; catalytic step 3).

### CROSS REFERENCE TO RELATED APPLICATIONS

This application is the U.S. National Stage under 35 U.S.C. §371 of International Patent Application No. PCT/US2007/63542 filed Mar. 8, 2007, which claims priority of U.S. Provisional Patent Application No. 60/780,120 filed Mar. 8, 2006, the disclosures of which are hereby incorporated herein by reference.

### STATEMENT REGARDING FEDERALLY SPONSORED RESEARCH OR DEVELOPMENT

The U.S. Government has a paid-up license in this invention and the right in limited circumstances to require the patent owner to license others on reasonable terms as provided for by the terms of Grant No. RO1 HL56712, awarded by the National Institutes of Health.

### BACKGROUND OF THE INVENTION

1. Technical Field

The present invention generally relates to methods and compositions for the prevention and treatment of vascular diseases, and more particularly to such compositions that provide cyclooxygenase (COX) and eicosanoid-synthesizing enzyme functions. Still more particularly the invention relates to the construction and expression of a single linked protein molecule that possesses both the enzyme functions of the native COX and that of PGIS.

2. Description of Related Art

### Prostanoids.

The recent discovery that cyclooxygenase (COX) isoform-2 (COX-2) inhibitors may be linked to heart disease has greatly increased the interest in understanding the biology of COX enzymes, which convert a lipid molecule, arachidonic acid (AA), into different prostanoids (part of the eicosanoid family) having diverse and/or opposite biological functions. FIG. 1 outlines the biosynthesis of prostanoids, comprising prostaglandins and the thromboxane, formed via the COX pathway from AA in three catalytic (tri-catalytic) steps (i-v): Arachidonic acid released from cell membrane phospholipids is converted to the prostaglandin G₂ (PGG₂, catalytic step i), and then to prostaglandin endoperoxide (prostaglandin H₂ (PGH₂)) (catalytic step ii) by COX isoform-1 (COX-1) or COX-2. PGH₂ is further isomerized to biologically active end-products (prostaglandin D₂ (PGD₂), E₂ (PGE₂), F₂ (PGF₂), and 12 (PGI₂ (prostacyclin)) or thromboxane A₂ (TXA₂)) by individual synthases (catalytic step iii) in tissue specific manners. Prostanoids act as local hormones in the vicinity of their production site to regulate hemostasis and smooth muscle functions. Unlike the stable level of COX-1 expression, COX-2 expression is inducible and it responds to the stimuli of pro-inflammatory and other pathogenic factors (vi). TXA₂ produced from PGH₂ by TXA₂ synthase (TXAS) has been implicated in various pathophysiological conditions as a proaggregatory and vasoconstricting mediator (vii-viii). PGI₂ is the main AA metabolite in vascular walls and has opposing biological properties to TXA₂, representing the most potent endogenous vascular protector acting as an inhibitor of platelet aggregation (ix) and a strong vasodilator on vascular beds (x-xiii). PGE₂ exhibits a variety of biological activities in inflammation (xiv). Aspirin and non-steroidal anti-inflammatory drugs (NSAID) inhibit both COX-1 and COX-2 activities to reduce the production of all prostanoids, which leads to thinning of the blood by reducing TXA₂ production and the suppression of inflammation through decreasing PGE2 production. The selective COX-2 inhibiting drugs exhibit anti-inflammatory effects similar to aspirin and NSAIDs, but they may also promote strokes and heart attacks by decreasing the production of PGI₂, and increasing the production of TXA₂. This may occur because, when the COX-2 enzyme was specifically inactivated by COX-2 inhibitors, the PGH₂ produced by COX-1 is still available to be converted into other prostanoids such as TXA₂ by TXAS, leading to an increased risk of thrombosis and vasoconstriction (vi).

### Protaglandin I₂

The function of PGI₂ is primarily mediated by the PGI₂ receptor (IP) on the cell surface. The role of PGI₂ as an endogenous anti-thrombotic and vasodilative agent was confirmed with the experimental data generated in IP receptor-knockout mice. The IP-deficient mice developed without vascular problems in normal situations. However, an increased thrombotic tendency was observed in the IP-deficient mice when endothelial damage was induced. These findings indicate that the anti-thrombotic system mediated by PGI₂ is activated in response to vascular injury to minimize the effects of vascular injury (1, 2, 12). It has been reported that defects in the IP receptor of platelets has pathogenetic significance for developing atherosclerosis at an early age. The evidence was derived from a 10 year-old human diagnosed with an occluded the left popliteal artery who also had a defect of her IP receptor. This defect appears to be genetically determined and to contribute to the development of atherosclerosis (4)

Recently, PGI₂ has also been determined to be a ligand for the nuclear hormone receptor PPAR. In 1990, Issemann & Green reported the discovery of a peroxisome proliferator-activated receptor (PPAR) (5). Since the initial report, three PPAR-isoforms, PPARα, β/δ and γ have been cloned and implicated in the regulation of the expression of genes involved in lipid metabolism. In both skeletal and cardiac muscle cells it has been demonstrated that the metabolic conversion of fatty acids is under controlled by PPARs. PGI₂ and PGI₂ agonists (e.g. carbaprostacyclin and iloprost), can effectively induce DNA binding and transcriptional activation by PPAR. PGI₂, acting as a ligand for PPARδ, induces increases expression of PPARδ in the arterial wall after balloon injury. This observation suggests that PGI₂ effects vasodilation and anti-platelet aggregation through the IP receptor and PPARδ (6). It has also been proposed that PGI₂, as a ligand for PPARδ, induces anti-inflammatory activity in vascular diseases, such as atherosclerosis. The proposed pathway for this anti-inflammatory activity is PGI₂ is bound by PPARδ, leading to the release of BCL-6, a transcriptional repressor of inflammation, by PPARδ. Release of BCL-6 may cause a decrease expression of inflammatory cytokine genes, reduced inflammation, and a decrease in atherosclerosis. Alternatively, in the absence of PGI₂, BCL-6 may remain bound by PPARδ, resulting in a pro-inflammatory response (7).

Recently, PGI₂ production in female reproductive tissues has been documented. PGI₂ is present in the embryo implantation site and human fallopian tubes (8, 9). The presence of PGI₂ at the site of implantation has lead to the proposal that PGI₂ plays an important role in the reproductive process in females, including egg transportation, embryo development, and implantation.

A selective increase in the production of PGI₂ with a decrease in TXA₂ production and PGE₂ production is the ideal model in (i) preventing and protecting against vascular diseases including inflammation, thrombosis, atherosclerosis, stroke, and heart attacks, and (ii) benefiting the reproductive process. From aspirin to the more recently developed COX-2 inhibitors, no drugs have yet to achieve this goal.

### SUMMARY OF PREFERRED EMBODIMENTS

The presently disclosed COX-2-linker-PGIS protein provides a way to specifically increase the biosynthesis of the vascular protector PGI₂, and is believed to be an important development in pharmacology. A recombinant 130-kDa protein was constructed by linking together human cyclooxygenase (COX) isoform-2 (COX-2) and prostacyclin synthase (PGIS) via 10 or 20 amino acid residues of a transmembrane sequence. As explained in the detailed description and drawings, the engineered protein was successfully expressed in human embryonic kidney cells (HEK293) and monkey COS-7 cells, and adopted the functions of COX and PGIS, which was able to continually convert arachidonic acid (AA) into prostaglandin G₂ (catalytic step 1), prostaglandin H₂ (catalytic step 2) and prostacyclin (PGI₂; catalytic step 3) even faster than the coupling reaction using unlinked, co-expressed COX-2 and PGIS.

Accordingly, certain embodiments of the present invention provide a new COX-2-linker-PGIS protein, and demonstrate that multiple catalytic activities of enzymes can be configured within a single protein molecule if the correct protein configuration is achieved. The new hybrid protein, with tri-catalytic activity, not only possesses the individual enzymes' activities, but has a faster turnover rate as compared to a mixture of separate COX-2 and PGIS enzymes.

In accordance with certain embodiments of the present invention, methods are provided for combining the enzymatic functions of COX-2 and PGIS in a single “hybrid” peptide molecule. In certain other embodiments, similar methods are employed to create other hybrid peptide molecules containing COX-1 or COX-2 linked with another downstream synthase, such as COX-linker-PGES, COX-linker-PGDS, and COX-linker-PGFS.

In accordance with certain embodiments of the present invention, a new generation of cDNA for COX gene therapy is provided, in which the cDNA encodes an above-described hybrid molecule. In accordance with other embodiments, a COX-linker-PGIS protein is used as a therapeutic reagent to instantly increase PGI₂ production locally through injection of the engineered protein. In this regard, it is of interest that COX-2 inhibitors inhibit COX-2 activity but not the COX-1 activity. Thus, the introduction of the COX-1-linker-PGIS hybrid protein to vascular systems is expected to help overcome the damage of the vascular functions caused by COX-2 inhibitors.

In accordance with certain embodiments of the present invention, a therapeutically useful hybrid protein is provided which comprises an engineered COX-linker-PGIS hybrid molecule. Given the importance of PGI₂ in vascular diseases and thrombosis, this hybrid protein is expected to be a useful, therapeutic molecule in vivo.

Certain embodiments of the present invention provide an isolated hybrid protein molecule comprising a cyclooxygenase (COX) amino acid sequence and an eicosanoid-synthesizing (ES) enzyme amino acid sequence with a linker sequence disposed therebetween and directly connecting the COX enzyme sequence to the ES enzyme sequence. In certain embodiments, the linker sequence is capable of functioning as a transmembrane linker in a cell such that the folding ability and function of each said enzyme is substantially unaltered compared to the folding ability and function of the respective native enzymes. In certain embodiments, the linker sequence is about 10 to 22 amino acids long. In certain embodiments, the linker sequence is His-Ala-Ile-Met-Gly-Val-Ala-Phe-Thr-Trp (SEQ ID NO. 1) or His-Ala-Ile-Met-Gly-Val-Ala-Phe-Thr-Trp-Val-Met-Ala-Leu-Ala-Cys-Ala-Ala-Pro-Pro-Leu-Val (SEQ ID NO. 2). In certain embodiments, the linker sequence is residues 1-11, 1-12, 1-13, 1-14, 1-15, 1-16, 1-17, 1-18, 1-19, 1-20 or 1-21 of SEQ ID NO. 2. In certain embodiments, the linker peptide provides approximately 10 Å separation between the catalytic sites of the COX and the ES enzyme. The connected enzymes are preferably capable of substantially normal folding and enzymatic activity compared to the native folding and enzymatic activity of the native COX and ES enzymes. In certain embodiments, the hybrid protein comprises cyclooxygenase, a transmembrane linker, and a prostacyclin synthase, and is chemically synthesized. In certain other embodiments, the hybrid protein comprises cyclooxygenase, a transmembrane linker, and a prostacyclin synthase, is recombinantly produced.

In accordance with certain embodiments of the present invention, a pharmaceutical composition is provided which comprises an above-described hybrid protein together with a pharmaceutically acceptable carrier.

Certain embodiments of the present invention provide an isolated DNA sequence encoding: a cyclooxygenase (COX), a transmembrane linker peptide, and an eicosanoid-synthesizing enzyme (ES). In some embodiments, the cyclooxygenase is a cyclooxygenase isoform-1 (COX-1). In some embodiments, the cyclooxygenase is a cyclooxygenase isoform-2 (COX-2). In some embodiments, the eicosanoid-synthesizing enzyme is a prostacyclin synthase (PGIS). In some embodiments, the linker sequence directly connects the COX sequence and the eicosanoid-synthesizing enzyme sequence, and has the above-described amino acid sequence.

Certain embodiments of the present invention provide a vector comprising an above-described DNA sequence. In some embodiments, the vector is an expression vector.

Some embodiments of the present invention provide a host cell containing an expressible DNA sequence encoding an above-described hybrid protein.

In accordance with another embodiment of the invention is provided a process for producing a hybrid protein that comprises a cyclooxygenase, a transmembrane linker, and a prostacyclin synthase. The process comprises culturing the above-described host cell under conditions suitable for expression of the DNA sequence encoding the hybrid protein molecule, and then recovering the biologically active hybrid protein molecule comprising a cyclooxygenase, a transmembrane linker, and a prostacyclin synthase. In some embodiments, the hybrid protein comprises enzymatically active cyclooxygenase, transmembrane linker, and enzymatically active prostacyclin synthase. In some embodiments, the process comprises, prior to the culturing step, transfecting the host cell with a vector comprising the DNA sequence.

Still further provided in accordance with certain embodiments of the invention is a method of treating an individual having a vascular disease, or at risk of developing a vascular disease. For example, the vascular disease may include one or more of the following conditions: stroke, heart attack, thrombosis, ischemia and inflammation in an organ or vessel of an individual's vascular system. The treatment method comprises administering to the individual an effective amount of the above-described pharmaceutical composition, to cause the production of at least one biologically active compound (e.g., PGI₂) that deters or prevents the occurrence of the vascular disease in the individual, or which ameliorates existing vascular disease, if present. In some embodiments, the step of administering comprises microinjecting the hybrid protein into at least one vascular cell in said individual.

Still another embodiment of the present invention provides a method of treating an individual having a vascular disease, or at risk of developing a vascular disease, in which the method comprises administering to the individual an effective amount of an above-described DNA sequence. Such administration causes the production of a above-described functional hybrid protein whereby at least one biologically active compound is produced that deters or prevents the occurrence of the vascular disease, or which ameliorates existing vascular disease, if present in the individual. The vascular disease may comprise one or more of the above-mentioned conditions in an organ or vessel of the individual's vascular system. In some embodiments, the step of administering comprises transfecting at least one vascular cell in the individual with a vector comprising the DNA sequence, or microinjecting the above-described vector into one or more vascular cell of the individual.

Still another embodiment of the present invention provides a method of reducing the adverse effects of a COX-2 inhibitor drug on vascular function. This method comprises administering to an individual in need thereof an effective amount of a hybrid protein which contains the structure COX-1-linker-PGIS. As a result, the COX-1 enzymatic activity of the hybrid protein counteracts at least some of the adverse vascular effects arising from COX-2 inhibitor administration to the individual. In some embodiments, the manner of administration comprises transfecting at least one vascular cell in the individual with a vector comprising a DNA sequence encoding the hybrid protein. In some embodiments, the manner of administration comprises microinjecting the hybrid protein into one or more vascular cell in the individual. These and other embodiments, features and advantages will be apparent with reference to the following detailed description and accompanying drawings.

### BRIEF DESCRIPTION OF THE DRAWINGS

FIG. 1 is an outline showing the biosynthesis of prostanoids through the coordination of the COX enzymes and their downstream synthases.

FIG. 2 is an illustration showing the engineered cDNA plasmids with single proteins containing a COX-2 sequence, a PGIS sequence, and a transmembrane linker sequence.

FIG. 3 depicts the western blot analysis of an overexpressed COX-2-linker-PGIS proteins in COS-7 cells (A) or HEK293 cells (B).

FIGS. 4A-B depict immunofluorescence micrographs of HEK293 cells. A: anti-human PGIS antibody. B: anti-human COX-2 antibody.

FIGS. 5A-B are graphs showing the tri-catalytic activities of the linked proteins directly converting AA to PGI2 using a High Performance Liquid Chromatography (HPLC) method, in which a: COX-2-22aa-PGIS. b: COX-2-10aa-PGIS. c: PGIS-10aa-COX-2. d: COX-2 and PGIS. e: untransfected cells.

FIGS. 6A-B are graphs showing the tri-catalytic activities of the linked proteins directly converting AA to PGI2 using Enzyme Immunoassay (EIA) analysis. (A) Expressed in COS-7. (B) Expressed in HEK293 cells.

FIG. 7 is a pair of graphs showing a comparison of the tri-catalytic activities of an engineered COX-2-linker-PGIS protein in the intact cells (A) compared to the membrane preparations (B).

FIG. 8 is a group of graphs showing the effects of a COX-2 inhibitor on the tri-catalytic activities of a representative COX-2-linker-PGIS.

FIG. 9 is a graph showing the enzyme kinetic properties of a representative COX-2-linker-PGIS with tri-catalytic properties.

FIG. 10 is a graph showing the time-course of the conversion of AA to PGI2 by representative COX-2-linker-PGIS proteins.

FIG. 11 is a flow diagram illustrating the subcloning of cDNA of the engineered COX-2-10aa-PGIS into the pcDNA3.1(+) vector using Hind III and Bgl II sites (A). Section B depicts agarose gel separation of the digested plasmid containing the COX-2-10aa-PGIS.

FIG. 12 is a map of the constructed Ad vector containing the cDNA sequence of COX2-10aa-PGIS fusion enzyme.

FIG. 13 is a bar graph showing the survival rate of HEK293 cells transfected with different amounts of recombinant Ad virus containing the cDNA of COX2-10aa-PGIS.

FIG. 14 is a pair of graphs showing the results of an activity assay of the fusion enzyme expressing in HEK293 cells using Ad-COX2-10aa-PGIS (A) and control Ad (B).

FIG. 15 is a Western blot analysis (A) for COX2-10aa-PGIS fusion enzyme expressed in the HEK293 cells, and a graph (B) showing the results of an enzyme activity assay for a representative recombinant COX2-10aa-PGIS expressed in HEK293 cells.

FIG. 16 is a group of four graphs showing the activity assay of the fusion enzyme expressing in COS-7 cells using 100 μL(A), 50 μL (B) and 10 μL (C) of the Ad-COX2-10aa-PGIS. The Ad virus without the fusion enzyme gene was used as control (D).

FIG. 17 is a Western blot analysis (A) for COX2-10aa-PGIS fusion enzyme expressed in the COS-7 cells, and a graph (B) showing the results of an enzyme activity assay for a representative recombinant COX2-10aa-PGIS expressed in COS-7 cells.

FIG. 18 is a set of fluorescence micrographs of CD34-positive SVF (A) and an ECL cell (B) differentiated from an adipocyte, demonstrating transient over-expression of COX-2-10aa-PGIS with an RFP tag. (C) is an SFV cell line with a stable expression of the COX-2-10aa-PGIS with the RFP tag and (D) is a non-transected SVF cell used as negative control (D).

FIG. 19 is a set of fluorescence micrographs showing ELC cells (A,B) and SVF (C,D) cells transfected with adenovirus containing GFP cDNA. A and C are green fluorescence photomicrographs, and B and D are regular light photomicrographs.

FIG. 20 is a graph showing the activity assay results for SVF cells transfected with adenovirus without (bar 1) and with (bar 2) COX2-10aa-PGIS cDNA.

FIG. 21 is a graph showing enzyme activity for the time course of the fusion enzyme expressed in the SVF cells using ad-COX2-10aa-PGIS.

FIG. 22 is a Western blot analysis of the fusion enzyme (CPX2-10aa-PGIS) expression using the viral vector (lanes 1-4) and non-viral vector (lane 6). Non-transfected SVF cells were used as negative control (lane 5).

FIG. 23 is a flow diagram showing the steps of subcloning COX2-10aa-PGIS cDNA into baculovirus vector.

FIG. 24 is a Western blot analysis of expressed COX2-10aa-PGIS fusion enzyme in different BV clones.

FIG. 25 is a graph showing the results of an activity assay for COX2-10aa-PGIS using HPLC.

FIG. 26 is a Western blot analyis of the time course for COX2-10aa-PGIS expression in a BV system.

FIG. 27 shows analyses of COX2-10aa-PGIS fusion enzyme by electrophoresis. (1) Coomassie Blue staining; (2) Western blot. The position of COX2-10aa-PGIS is indicated by an arrow.

FIG. 28A is a graph showing the initial velocity of the PGI2 biosynthesis by COX2-10aa-PGIS expressed in HEK293 cells.

FIG. 28B is a graph showing the initial velocity of PGI2 biosynthesis by co-expressing individual COX2 and PGIS.

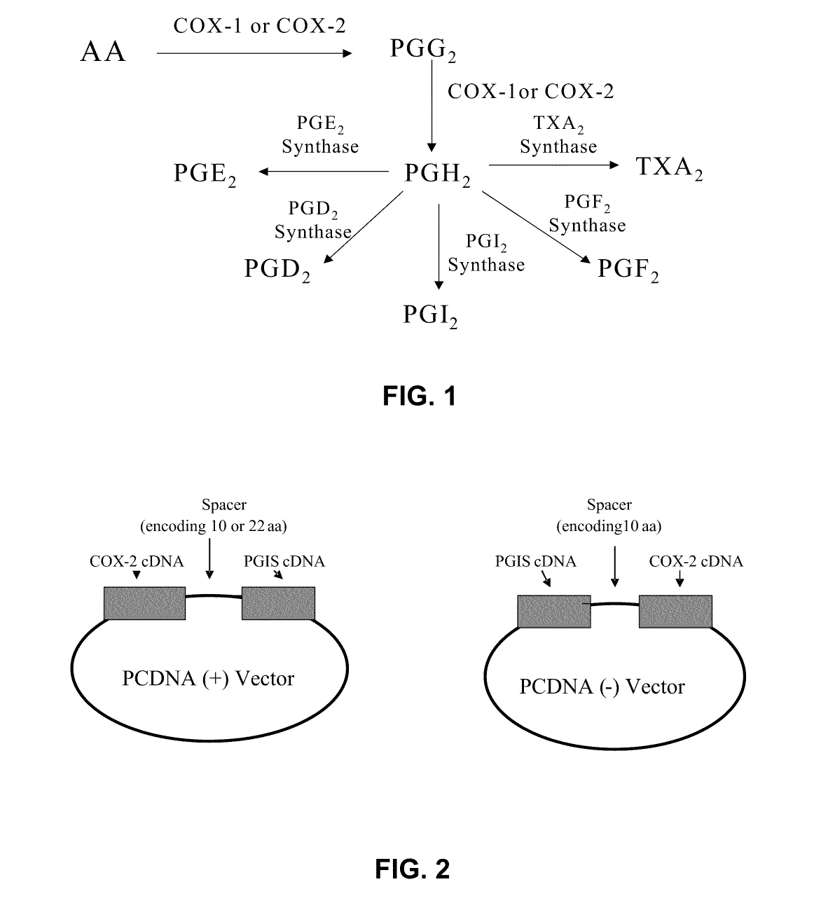

### DETAILED DESCRIPTION OF THE PREFERRED EMBODIMENTS

Recent studies of the structure and function relationship of COX enzymes and PGIS have advanced our knowledge of the molecular mechanisms involved in the biosynthesis of PGI₂ in native cells. Crystallographic studies of detergent-solubilized COX-1 and COX-2 suggest that the catalytic domains of the proteins lie on the luminal side of the endoplasmic reticulum (ER) and are anchored to the ER membrane by the hydrophobic side chains of the amphipathic helices A-D. These hydrophobic side chains of the putative membrane anchor domains also form an entrance to the substrate-binding channel and potentially form an initial docking site for the lipid substrate, AA (13-14). Recent progress in the topology and structural studies of human PGIS and TXAS have led to the proposal of models in which PGIS and TXAS have catalytic domains on the cytoplasmic side of the ER, opposite the orientation of COXs (15-17). In this configuration, the substrate channels of all the three enzymes, COX, PGIS and TXAS, open at or near the ER membrane surface. The coordination between COXs and PGIS or TXAS in the biosynthesis of TXA₂ and PGI₂ may be facilitated by the enzyme's anchoring in the lipid membrane (18). The physical distances between COXs and PGIS are very small. It was hypothesized that it is possible to create a single protein molecule containing COX and PGIS sequences with minimum alteration of both enzymes' folding and membrane topologies by extending the N-terminal membrane anchor domain of PGIS using a transmembrane sequence linked to the COX-1 or COX-2, which then adopts the functions of both enzymes of COX and PGIS. In this case, AA can be directly converted into the vascular protector, PGI₂, with decreases of the unwanted PGE₂ and TXA₂ productions.

The studies described herein are the first to demonstrate that a single, linked protein molecule can perform triple enzyme catalytic functions. One highly desirable feature of certain embodiments of the invention is that the linked protein allows for the direct synthesis of the potent vascular protector, PGI₂, from AA with a high efficiency, which may be used to prevent and rescue patients from vascular disease, including strokes, heart attacks, thrombosis, and ischemia through specifically increasing PGI₂ production. Gene delivery of the engineered cDNA and/or giving the engineered protein to tissues in vivo is one means for accomplishing these benefits. The discovery also provides evidence that the COX enzymes can be linked with other downstream eicosanoid-synthesizing (ES) enzymes, thereby generating other linked proteins, to specifically regulate the biosynthesis of eicosanoids, which play diverse and potent biological functions in human.

### Example 1

### Preparation of Linked Protein

To test this hypothesis, a linker with 10 (His-Ala-Ile-Met-Gly-Val-Ala-Phe-Thr-Trp (10aa)) or 22 (His-Ala-Ile-Met-Gly-Val-Ala-Phe-Thr-Trp-Val-Met-Ala-Leu-Ala-Cys-Ala-Ala-Pro-Pro-Leu-Val (22aa)) residues of the structurally defined helical transmembrane domain of bovine rhodopsin were used to configure the engineered cDNA containing the COX-2 and PGIS sequences. The sequence begins at the N-terminus of COX-2, which is linked to either DNA encoding 10aa (COX-2-10aa-PGIS) or DNA encoding 22aa (COX-2-22aa-PGIS), and then these are linked to the N-terminus of PGIS that ends with the C-terminus of PGIS. In contrast, an engineered cDNA containing reversed sequences from PGIS, DNA encoding 10aa to COX-₂, were also prepared as a control (PGIS-10aa-COX-2 (FIG. 2).

Engineered cDNA plasmids with single genes encoding the COX-2 or PGIS sequences. COX-2 linked to PGIS, and PGIS linked to COX-2 through the 10aa or 22aa sequence were generated by PCR approach (19) and subcloning procedures provided by the vector company (Invitrogen). Briefly, the corresponding cDNA sequences were isolated from the pSG5 or pcDNA3.1 vector containing human COX-2 or PGIS by PCR using the primers containing the DNA encoding 10aa or DNA encoding 22aa and KpnI or Bam HI cutting sites at both end (the 5′ end of the anti-sense primer was connected with the DNA sequences of the designed linker). The resulting cDNA segment was cut with the corresponding restriction enzymes and ligated into the corresponding sites at the pcDNA3.1 vector.

### Example 2

### Detection of Linked Protein

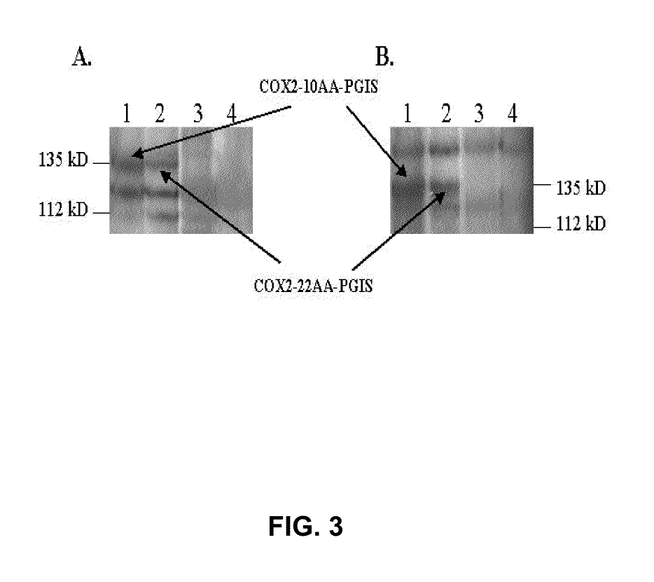

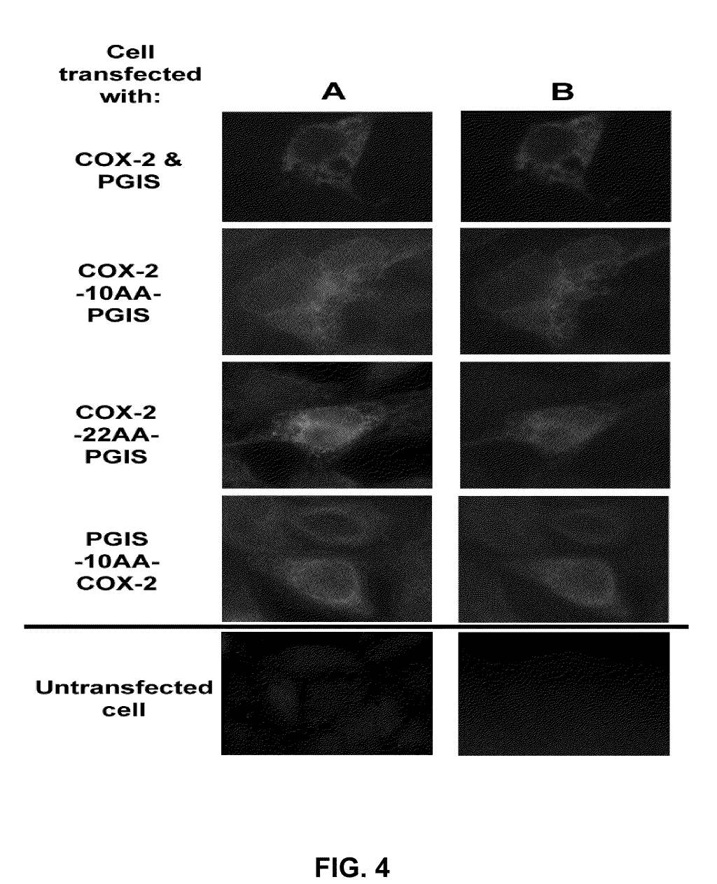

The cDNAs of the engineered COX-2-10aa-PGIS, COX-2-22aa-PGIS, and PGIS-10aa-COX-2 were successfully cloned into a pcDNA3.1 vector containing a cytomegalovirus early promoter using a PCR (polymerase chain reaction) cloning approach (FIG. 2). In FIG. 2, engineered cDNA plasmids with single proteins containing COX-2 and PGIS sequences are shown. COX-2 linked to PGIS, and PGIS linked to COX-2 through the 10aa or 22aa sequence were generated by PCR approach (19) and subcloning procedures provided by the vector company (Invitrogen). Briefly, the corresponding cDNA sequences were isolated from the pSG5 or pcDNA3.1 vector containing human COX-2 or PGIS by PCR using the primers containing the 10aa or 22aa and KpnI or Bam HI cutting sites at both ends (the 5′ end of the anti-sense primer was connected with the DNA sequences of the designed linker). The resulting cDNA segment was cut with the corresponding restriction enzymes and ligated into the corresponding sites at the pcDNA3.1 vector. The correct sequences were confirmed by DNA sequencing and endonuclease digestion analyses. The expression of the recombinant proteins was tested in COS-7 cell line (monkey epithelial cells) and HEK293 cell line (human embryonic kidney 293 cells) by a transient protein expression approach using Lipofectamin200 (Invitrogen). COX-2-10aa-PGIS, and COX-2-22aa-PGIS were successfully overexpressed in the both cell lines having a molecular weight of approximately 130 kDa as demonstrated by Western Blot analysis (FIGS. 3A,B). In contrast, the expressed PGIS-10aa-COX-2 protein was undetected by Western Blot indicating that it may have been degraded due to the lack of a correct protein folding. FIGS. 3A,B show a Western blot analysis of the overexpressed recombinant proteins in COS-7 (A) or HEK293 (B) cells. The procedures were described previously (15-17). Briefly, COS-7 or HEK293 cells were grown for 24 hours to 90-95% confluent and then transfected with a purified cDNA plasmid (24 μg/dish (100 mm)) by the Lipofectamin™2000 method (20) following the manufacturer's instructions (Invitrogen). For the co-transfection, the cells were transfected with 12 μg of human COX-2 cDNA plasmid and 12 μg of human PGIS cDNA plasmid. Approximately 48 hours after transfection, the cells were harvested for Western blot analysis. 20 μg of the proteins of the cells transfected with the cDNA(s) of COX-2-10aa-PGIS (lane 1), COX-2-22aa-PGIS (lane 2), PGIS-10aa-COX-2 (lane 3) or untransfected cells (lane 4) were subjected to SDS-PAGE and then transferred onto a nitrocellulose membrane. The membrane was probed with rabbit anti-PGIS peptide antibody (A) or anti-COX-2 peptide antibody (B) and then stained with horseradish peroxidase-labeled goat-anti rabbit antibody. Molecular weight standards were run, and the lines on the left and right show the positions of the these standards. The engineered proteins are indicated with arrows.

### Example 3

### Localization of Linked Protein to Endoplasmic Reticulum Membrane

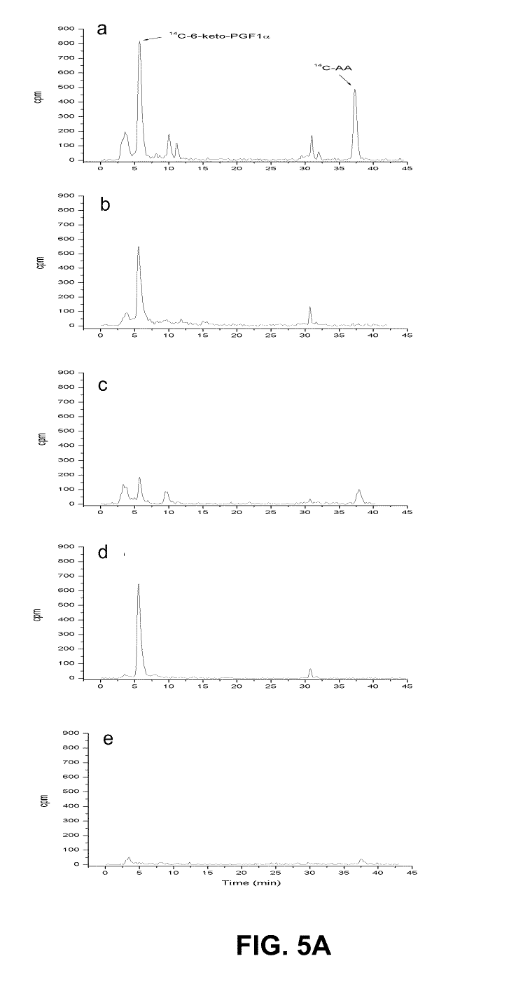

To test whether the expressed COX-2-10aa-PGIS and COX-2-22aa-PGIS could anchor to the ER membrane, adopting similar membrane topologies as the native COX-2 and PGIS, fluorescence immunostaining was used to localize the subcellular distribution of the engineered enzymes overexpressed in COS-7 cells. A similar pattern of ER staining was clearly observed in the COS-7 cells expressing the engineered proteins compared to the cells co-expressing the individual COX-2 and PGIS using either anti-human PGIS (FIG. 4A) or anti-human COX-2 (FIG. 4B) antibodies. Low-level expression of PGIS-10aa-COX-2 was also observed in the transfected cells, (FIGS. 4A,B). The correct pattern of ER staining indicates that the engineered COX-2-10aa-PGIS and COX-2-22aa-PGIS have correct folding and ER membrane anchoring functions and are more suitable for enzymatic activity compared to the PGIS-10aa-COX-2 engineered protein.

In FIGS. 4A,B, immunofluorescence micrographs of HEK293 cells are shown. The general procedures for the indirect immunostaining were described previously (15-16, 21). In brief, the cells were grown on cover-slides and transfected with cDNA plasmid(s) as described in FIG. 3. The cells were generally permeabilized by saponin, and then incubated with the affinity-purified rabbit anti-PGIS peptide antibody (A) (16) or mouse anti-COX-2 antibody (B). The bound antibodies were incubated with FITC-labeled goat-anti rabbit IgG (A) or Rhodomin-labeled rabbit anti-mouse IgG (B). The FITC-labeled goat-anti rabbit IgG (A) or Rhodomin-labeled rabbit anti-mouse IgG were examined by fluorescence microscopy (16).

### Example 4

Detection of PGI₂

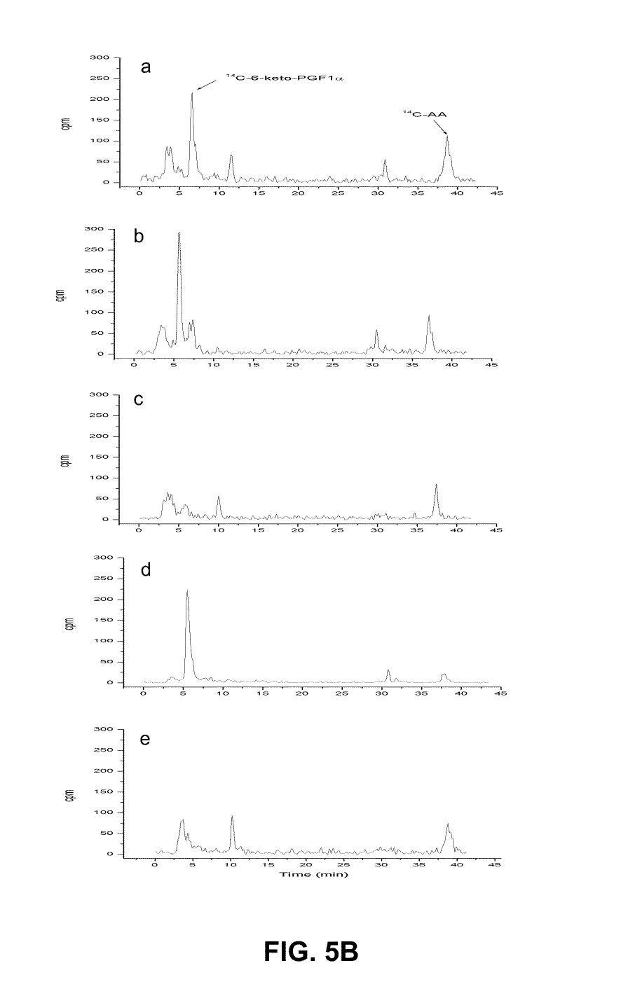

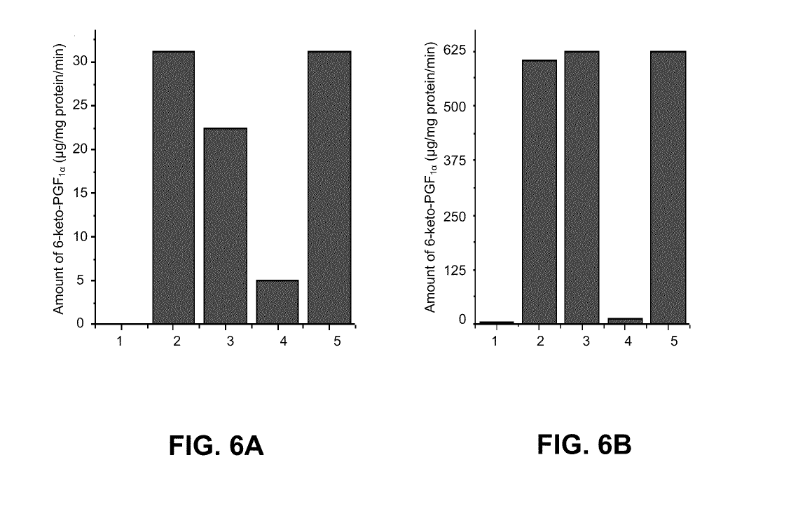

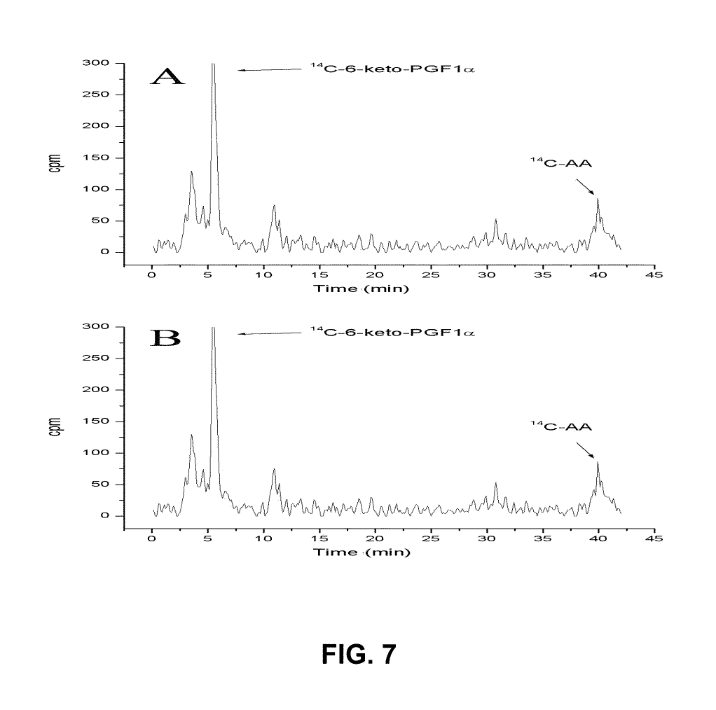

Evidence for the conversion of AA into the vascular protector PGI₂ by the tri-catalytic reactions in a single protein of COX-2-10aa-PGIS or COX-2-22aa-PGIS were given by two independent assays, High Performance Liquid Chromatography (HPLC) analysis (FIG. 5A and FIG. 5B), and Enzyme Immunoassay (EIA, FIGS. 6A,B) using AA as a substrate for the cells overexpressing the engineered proteins. The tri-catalytic activities in the engineered single COX-2-10aa-PGIS or COX-2-22aa-PGIS proteins were identical to the reactions mediated by co-expression of COX-2 and PGIS in the cells (FIGS. 5A, 5B, and 6A,B). It should be noted that the synthesized PGI₂ was unstable and quickly degraded into a stable 6-keto-PGF_{1α}. Thus, the level of 6-keto-PGF_{1α} shown in the HPLC and EIA represents the amounts of PGI₂ produced by the enzymatic reactions (FIGS. 5A, 5, 6A-B). COX-2-10aa-PGIS and COX-2-22aa-PGIS expressed in the intact cells exhibited very similar tri-catalytic activities in the direct conversion of AA to PGI₂. The similar catalytic activities were also observed for the membrane protein preparations as compared to the intact cells (FIG. 7, graphs A and B). Thus, the COX-2-10aa-PGIS was used as an example of enzyme properties of COX-2-10aa-PGIS and COX-2-22aa-PGIS.

Determination of the tri-catalytic activities of the recombinant proteins directly converting AA to PGI₂ using a HPLC method. The cells (˜0.1×10⁶) transfected with the recombinant cDNA(s) of COX-2-22aa-PGIS (A), COX-2-10aa-PGIS (B), PGIS-10aa-COX-2 (C) and COX-2 and PGIS (D) as described in FIGS. 3A,B, or the untransfected cells (E) were washed three times, suspended in 0.01 M phosphate buffer, pH 7.2 containing 0.15% NaCl (PBS) and then incubated with [¹⁴C]-AA (10 μM) in a total volume of 0.1 ml. After five min., the reaction was terminated by addition of 0.5 ml of 0.1% acetic acid containing 30% acetonitrile (buffer A), and centrifuged at 12,000 rpm for 10 min. The supernatant was separated by HPLC on a C18 column (4.5×250 mm) using buffer A with a gradient of 30-80% acetonitrile. The [¹⁴C]-AA metabolites were determined by a liquid scintillation analyzer built into the HPLC system. The retention time of [¹⁴C]-6-keto-PGF_{1α} and [¹⁴C]-AA were calibrated by standards under the same conditions. The amount of 6-keto-PGF_{1α} produced represents the amount of PGI₂ produced.

Determination of the tri-catalytic activities of the recombinant proteins directly converting AA to PGI₂ using Enzyme Immunoassay (EIA). After the enzyme reactions as described in FIGS. 5A and 5B, the reaction mixtures (1. Untransfected cells, 2. COX-2-22aa-PGIS, 3. COX-2-10aa-PGIS, 4. PGIS-10aa-COX-2, and 5. Co-expressed COX-2 and PGIS) were diluted 100 times with PBS containing 0.1% BSA, and then used for quantitative determination of 6-keto-PGF_{1α} using an EIA kit following the instructions of the manufacture (Cayman Chemical, Ann Arbor, Mich.).

Comparison of the tri-catalytic activities of the engineered COX-2-10aa-PGIS in the cells and in the membrane preparation. The tri-catalytic activities of COX-2-10aa-PGIS expressed in COS-7 cells were determined using the intact cells (A) as described in FIGS. 5A and 5B. The same amount of cells was homogenized and the total membrane protein was collected by ultracentrifugation and used for the activity assay (B). The amount of 6-keto-PGF_{1α} represents the amount of PGI₂ produced.

In FIGS. 5A and 5B the determination of the tri-catalytic activities of the recombinant proteins directly converting AA to PGI₂ using a HPLC method is shown. The cells (˜0.1×10⁶) transfected with the recombinant cDNA(s) of COX-2-22aa-PGIS (A), COX-2-10aa-PGIS (B), PGIS-10aa-COX-2 (C) and COX-2 and PGIS (D) as described in FIGS. 3A,B or the untransfected cells (E) were washed three times, suspended in 0.01 M phosphate buffer, pH 7.2 containing 0.15% NaCl (PBS) and then incubated with [¹⁴C]-AA (10 μM) in a total volume of 0.1 ml. After five minimums, the reaction was terminated by addition of 0.5 ml of 0.1% acetic acid containing 30% acetonitrile (buffer A), and centrifuged at 12,000 rpm for 10 min. The supernatant was separated by HPLC on a C18 column (4.5×250 mm) using buffer A with a gradient of 30-80% acetonitrile. The [¹⁴C]-AA metabolizes were determined by a liquid scintillation analyzer built in the HPLC system. The retention time of [¹⁴C]-6-keto-PGF_{1α} and [¹⁴C]-AA were calibrated by standards under the same conditions. The amount of 6-keto-PGF_{1α} produced represents the amount of PGI₂ produced.

In FIGS. 6A-B, the determination of the tri-catalytic activities of the recombinant proteins directly converting AA to PGI₂ using Enzyme Immunoassay (EIA) is shown. After the enzyme reactions as described in FIGS. 5A and 5B, the reaction mixtures (1. Untransfected cells, 2. COX-2-22aa-PGIS, 3. COX-2-10aa-PGIS, 4. PGIS-10aa-COX-2, and 5. Co-expressed COX-2 and PGIS were diluted 100 times with PBS containing 0.1% BSA, and then used for quantitative determination of 6-keto-PGF_{1α} using an EIA kit followed the instructions of the manufacture (Cayman Chemical, Ann Arbor, Mich.).

FIG. 7, graphs A and B, show a comparison of the tri-catalytic activities of the engineered COX-2-10aa-PGIS in the cells and in the membrane preparation. The tri-catalytic activities of COX-2-10aa-PGIS expressed in COS-7 cells were determined using the intact cells (A) as described in FIGS. 5A and 5B. The same amount of cells was homogenized and the total membrane protein was collected by ultracentrifugation and used for the activity assay (B). The amount of 6-keto-PGF_{1α} produced represents the amount of PGI₂ produced.

### Example 5

### Biological Activity of Linked Protein

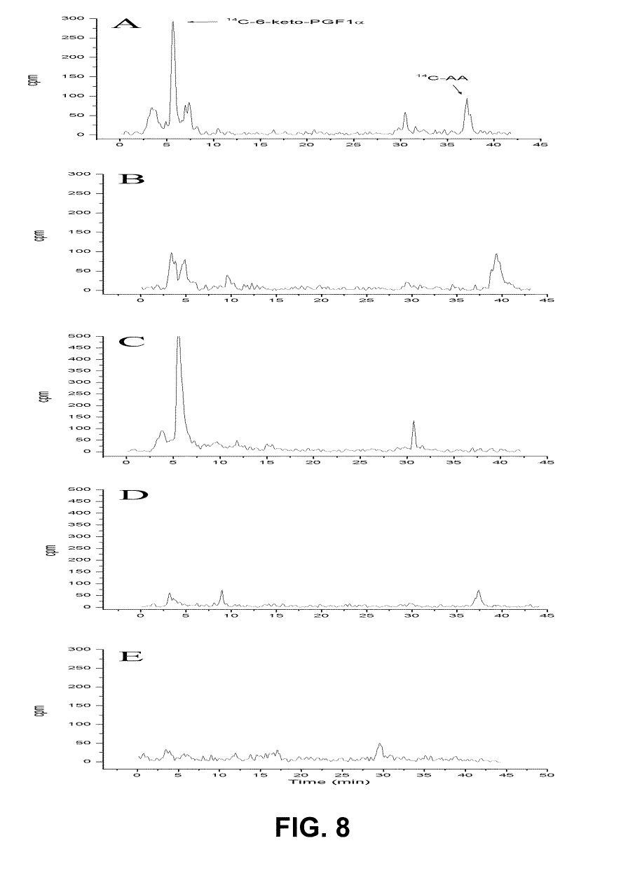

The biological activity of the COX-2-10aa-PGIS molecule directly converting [¹⁴C]-AA to [¹⁴C]-PGI₂ was further confirmed by an inhibition assay using a COX-2 inhibitor, NS-398, and a PGIS inhibitor, U46619 (FIG. 8).

The effects of the COX-2 inhibitor on the tri-catalytic activities of COX-2-10aa-PGIS are shown in FIG. 8, which demonstrates the conversion of AA to PGI₂ by the COX-2-10aa-PGIS overexpressed in COS-7 (A, B and E) and in HEK293 (C and D) cells in the absence (A and C) and presence (B and D) of a COX-2 inhibitor, NS-398 (60 μM), or in the presence of a PGIS inhibitor, U46619 (60 μM, E), using the HPLC method as described in FIGS. 5A and 5B.

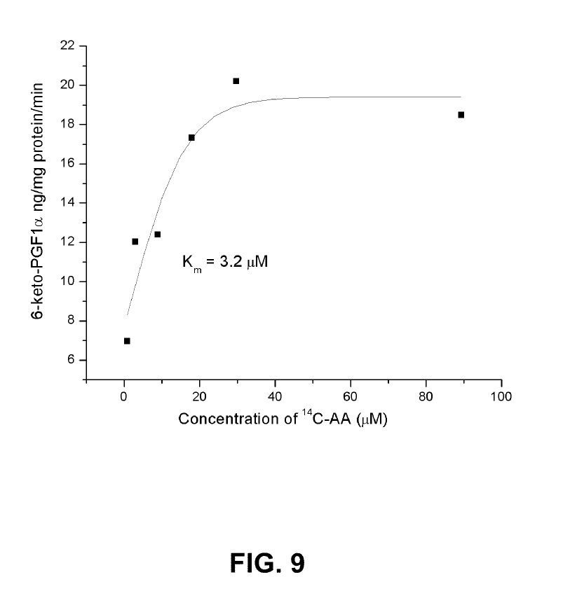

FIG. 9 shows the enzyme kinetic properties of the COX-2-10aa-PGIS with the tri-catalytic activities. Different concentrations of AA was added to the membrane preparations of the COS-7 cells overexpressed COX-2-10aa-PGIS protein (˜20 μg), after the incubations for five minutes, the degraded PGI₂ product, 6-keto-PGF_{1α}, was determined by EIA method as described in FIGS. 6A-B. The membrane preparation of the untransfected COS-7 cells was used as controls. The detailed kinetic studies revealed that the engineered COX-2-10aa-PGIS has a Km value (˜3.2 μM, FIG. 9) similar to the reported Km values for the individual COX-2 and PGIS enzymes. These data demonstrate that the engineered COX-2-linker-PGIS adopts the full enzymatic activities of both native COX-2 and PGIS. The tri-catalytic activities in converting AA to PGG₂ and ultimately to PGH₂ and to PGI₂ can be completed by the engineered single protein.

Enzyme kinetic properties of the COX-2-10aa-PGIS with the tri-catalytic activities. Different concentrations of AA was added to the membrane preparations of the COS-7 cells overexpressed COX-2-10aa-PGIS protein (˜20 μg), after the incubations for five minimums, the degraded PGI₂ product, 6-keto-PGF_{1α}, was determined by EIA method as described in FIGS. 6A-B. The membrane preparation of the untransfected COS-7 cells was used as controls.

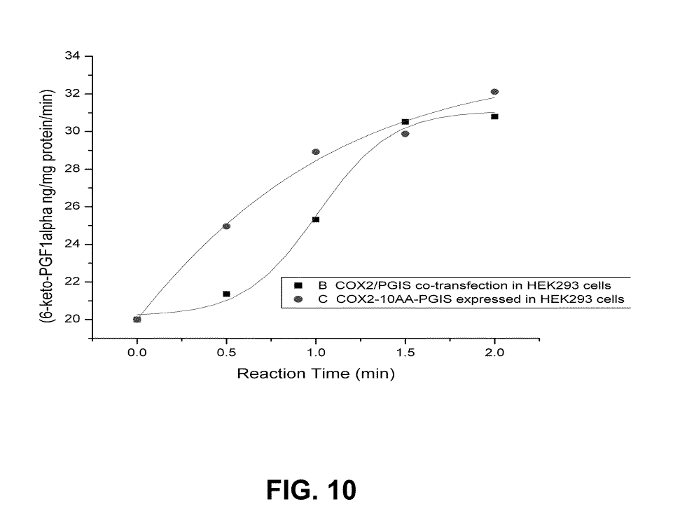

Additionally, in time course studies, the tri-catalytic turn-over rate of the COX-2-10aa-PGIS in the first 0.5-1 min reaction was faster than the reactions using the two enzymes of COX-2 and PGIS co-expressed (FIG. 10). Time-course of the conversion of AA to PGI₂ by the recombinant proteins is shown in FIG. 10. Referring still to FIG. 10, the conversion of AA to PGI₂ by the COX-10aa-PGIS () or the co-expressed COX-2 and PGIS in the HEK293 cells (▪) were performed with different reaction times using the EIA method as described in FIGS. 6A-B. The amounts of the produced PGI₂ (6-keto-PGF_{1α}) at the different reaction times were plotted. The untransfected cells were used as controls.

The results demonstrate that the engineered molecules can compete with the endogenous COX-downstream synthases to convert COX-generated PGH₂ to PGI₂. Thus, the engineered COX-2-linker-PGIS not only adopts the COX and PGIS activities but also increases the selectivity of converting AA to PGI₂. This is of particular importance in pathophysiological conditions, in which quick conversion of AA or PGH₂ to PGI₂ will reduce the substrate available to other COX-downstream synthases such as TXAS and PGES. Incorporating either of the engineered proteins in vivo will cause a decrease in the biosynthesis of TXA₂ and PGE₂ in the cells, which will be important for preventing and reversing thrombosis, hypertension, and ischemic tissues resulting from TXA₂ and for the reduction of vascular inflammation stimulated by PGE₂. The cDNA of the COX-2-linker-PGIS may be used as a gene therapy reagent to prevent and treat the thrombosis associated with strokes and heart attacks. In addition, the biologically active COX-2-linker-PGIS protein may be used as a reagent by injection into tissues to rapidly synthesize PGI₂ in vivo.

The new COX-2-linker-PGIS proteins offer many advantages, including its unique protein design has demonstrated that multiple catalytic activities of enzymes can be configured within a single protein molecule if the correct protein configuration was achieved (10). It is demonstrated that the new hybrid protein, with tri-catalytic activity, not only possesses the individual enzymes' activities, but has a faster turnover rate as compared to a mixture of the parent enzymes (11). Since COX-1 shares similar molecular features with COX-2, the method used for preparation of COX-2-linker-PGIS is also suitable for general preparation of the COX-1-linker-PGIS molecule, or any other prostaglandin synthase (12). The methods and information used to combine the enzymatic functions of COX-2 and PGIS may also be used to create single “hybrid” peptide molecules containing COX-1 or COX-2 linked with any downstream synthase, such as COX-linker-PGES, COX-linker-PGDS, and COX-linker-PGFS (13).

Successfully creating the functional COX-2-linker-PGIS peptide has settled several important controversies in COX biology. For decades it was unknown whether delivery of PGH₂ from COXs to their downstream synthases required a carrier protein (10). The present disclosure demonstrates that the process of PGH₂ moving from COX-2 to PGIS can be achieved within a single molecule and suggested that the process does not need a carrier protein. The concept that the enzymatic activities of COXs occur under the conditions of the defined dimerization structures has been generally adopted because the studies of crystal structures for detergent solubilized COX-1 and COX-2 revealed that the active enzymes were in dimer forms (11). This concept is not fully supported by current studies because it is highly probable that the engineered protein has different dimerization features relative to the native COX enzymes. For decades, the exact physical distances between COXs and their downstream synthases in the cells during the biosynthesis of prostanoids were unknown (12). The present studies clearly demonstrate that the distance between the two enzymes must be very close because the turn over rate of the isomerization of PGH₂ to PGI₂ in the cells that co-expressed COX-2 and PGIS is very similar to the single molecule of COX-2-10aa-PGIS which has an approximately 10 Å separation between the catalytic sites of COX-2 and PGIS (FIG. 10).

### Example 6

Subcloning the cDNA Encoding the Engineered COX-2-10aa-PGIS Gene into Adeno-Associated Virus Vector

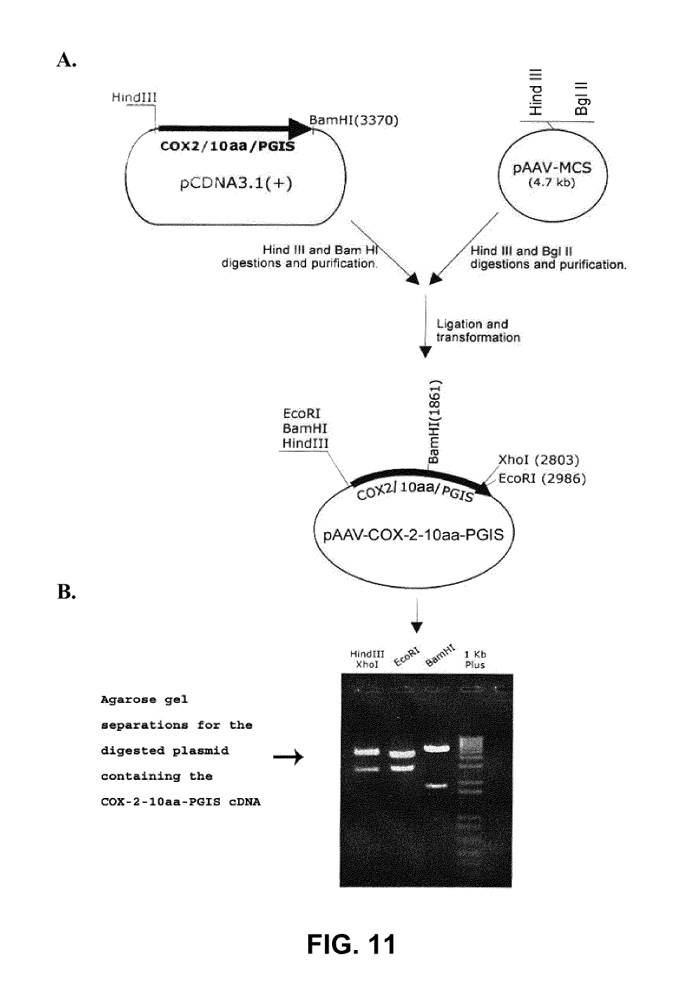

Adeno-associated virus (AAV) vector is one of the best viral vectors for gene transfer into cells and tissues in vitro and in vivo. In order to establish an effective gene transfer system for the COX-2-10aa-PGIS used for ischemic therapy in vivo, the cDNA of the engineered COX-2-10aa-PGIS in the pcDNA3.1(+) vector was subcloned into pAAV-MCS vector from AAV Helper-Free system (Stratagene, Cedar Creek, Tex.) using Hind III and Bgl II sites (pAAV-COX-2-10aa-PGIS, FIG. 11A). The successful cloning was confirmed by endonuclease digestion analyses (FIG. 11B), and the correct sequence was further confirmed by DNA sequencing.

Preparation of the Primary pAAV-COX-2-10aa-PGIS Viral Stock

The viral stock of pAAV-COX-2-10aa-PGIS was prepared following the manufacturer's instructions (Stratagene). Briefly, the AAV-293 cells were plated in 100 mm tissue culture plates with 10 ml of DMEM growth medium 48 hours prior to transfection until they were approximately 70-80% confluent. 10 μg of pAAV-COX-2-10aa-PGIS was co-transfected with two other plasmids (10 μg) provided from the system: pAAV-RC and pHelper. 1 ml of 0.3M CaCl₂ was added to the three plasmids and mixed gently. The mixture was then added to 1 ml 2×HBS drop wise and mixed gently. The DNA/CaCl₂/HBS suspension was immediately applied to the plate of cells drop wise and swirling gently to distribute the suspension evenly in the medium. The plates were incubated at 37° C. for 6 hours and the medium was then replaced with 10 ml fresh DMEM growth medium. After incubating for an additional 66-72 hours at 37° C., the cells with DMEM growth medium were scraped into the tube and subjected to four rounds of freeze/thaw by alternation of the tube between the dry ice-ethanol bath and the 37° C. water bath (with brief vortexing after each thaw). The cellular debris was collected by centrifugation at 10,000×g for 10 min and the supernatant (primary pAAV-COX-2-10aa-PGIS virus stock) was stored at −80° C. The virus stock was then concentrated and purified using the AAV-Pure™ Maxi AAV purification kits (Biovintage, San Diego, Calif.). The pAAV-COX-2-10aa-PGIS viral stock is readily available in the inventor's laboratory to infect cells/tissues in vitro and in vivo.

### Example 7

Construction and High Expression of an Engineered Fusion Enzyme with Triple Catalytic Functions Directly Converting Arachidonic Acid to the Vascular Protector Prostacyclin (PGI2) Using an Adenovirus System

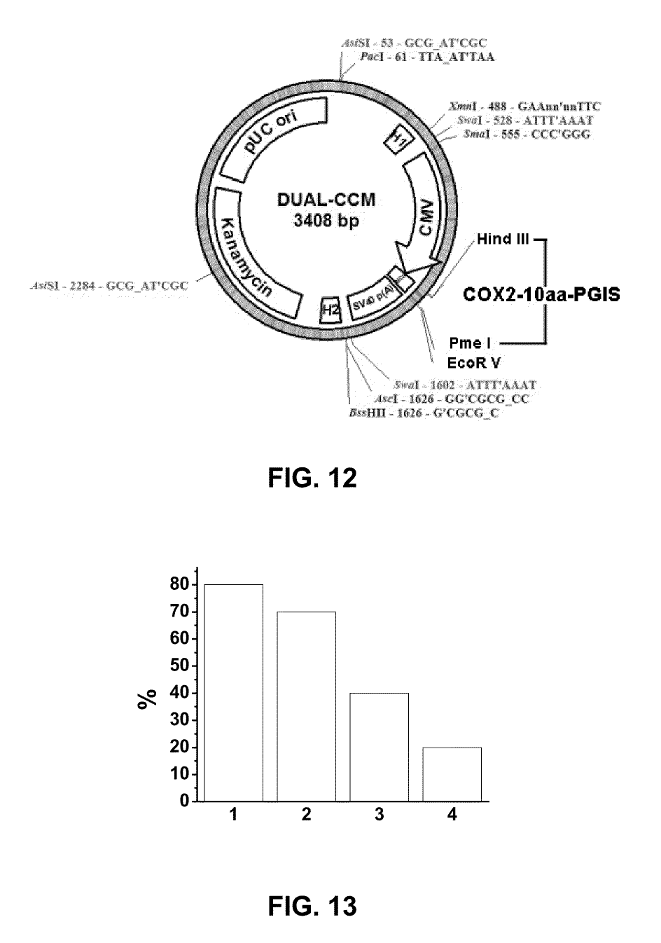

Construction of the cDNA of the newly engineered COX2-10aa-PGIS fusion enzyme into adenovirus (Ad) vector. The cDNA of the newly engineered active fusion enzyme linking human COX2 and PGI2 synthase was subcloned into an Ad vector. The map used for cloning is shown in FIG. 12. The Hind III/Pme I fragment of pcDNA3.1(+)-COX2-10aa-PGIS was cloned into the Hind III/Eco RV site of the DUAL-CCM vector (from Vector BioLabs), in which the expression of COX2-10aa-PGIS is under the control of a CMV promoter. The resultant expression cassette (CMV-COX2-10aa-PGIS-poly(A)) was transferred into the E1/E3-deleted Ad backbone DNA through homologous recombination. For Ad packaging, the positive recombinant viral DNA was linearized through Pac I digestion, and transfected into HEK293 cells. The recombinant Ad virus with the COX2-10aa-PGIS gene (Ad-COX2-10aa-PGIS) produced in the cells was used for further studies.

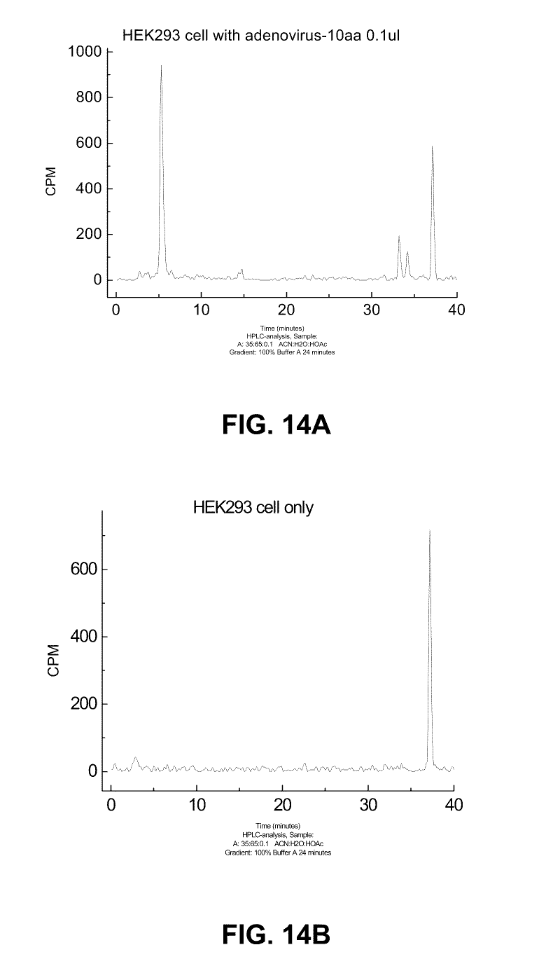

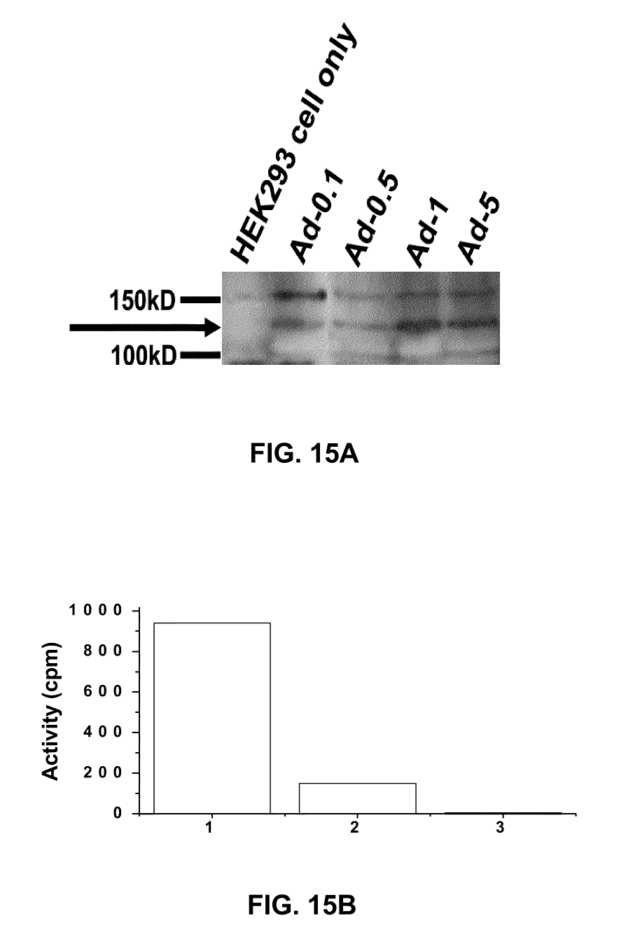

Expression of the fusion enzyme in HEK293 cells using Ad-COX2-10aa-PGIS. The expression of the fusion enzyme using Ad-COX2-10aa-PGIS was first tested in HEK293 cells. The HEK293 cells were cultured in 6-well plates and then transfected with different amounts of the recombinant adenovirus containing the cDNA of the fusion enzyme. HEK293 cells were able to amplify the virus and successfully express the fusion enzyme. However, with increasing viral amounts, the viability of the cells was greatly reduced. Thus, finding the cell survival rate for the viral transfection in these HEK 293 cells is the first step toward optimizing the expression conditions for the fusion enzyme. FIG. 13 shows the cell survival rate of the HEK293 cells transfected with Ad-Cox2-10aa-PGIS. Values from 0.1 to 1 μL per well of the viral stock were the most suitable for transfection (FIG. 13). HEK239 cells cultured in a 6-well plate at 90% confluence were transfected with Ad-COX2-10aa-PGIS using 0.1 μL (1); 0.5 μL (2); 1 μL (3) and 5 μL (5) of the viral stock. The expression of the fusion enzyme in the transfected HEK293 cells using different amounts of the viral stock was confirmed by activity assays (FIG. 14) and western blot analysis (FIG. 15A). FIG. 15 (A) is a Western blot analysis for COX2-10aa-PGIS fusion enzyme expressed in the HEK293 cells. The expressed fusion enzyme in the transfected cells using 0. 1 to 5 μL of the viral stock were separated by 7% PAGE and then analyzed by Western blot using anti-PGIS antibody. The expressed fusion enzyme is indicated with an arrow. (B) A bar graph shown the results of an enzyme activity assay for the recombinant COX2-10aa-PGIS expressed in HEK293 cells. The enzyme assay was performed by addition of [14C]-AA to the HEK293 cells transfected with the viral system-Ad-COX2-10aa-PGIS (1) and non-viral system-pcDNA3.1 vector/Lipofectamine (2). The [14C]-6-keto-PGF1α (degraded [14C]-PGI2) product was measured in CPM by a scintillation analyzer connected to an HPLC-system using a C18-column. The untransfected HEK293 cells were used as a negative control (3).

It is clear that with increasing viral amounts, the transfection rates also increased (FIG. 15A). However, it should be noted that the cell death rate also increased with increasing concentrations (FIG. 13). Further studies revealed that the best expression of the fusion enzyme in the HEK293 cells occurs in the presence of 1.0 μL of the virus stock (FIG. 15A). This amount was able to produce the fusion enzyme in the cells with approximately 6-folds higher than that of the expression system using the pcDNA3.1 vector (FIG. 15B). The data indicates that Ad-COX2-10aa-PGIS is a high expression system for the fusion enzyme in HEK293 cells.

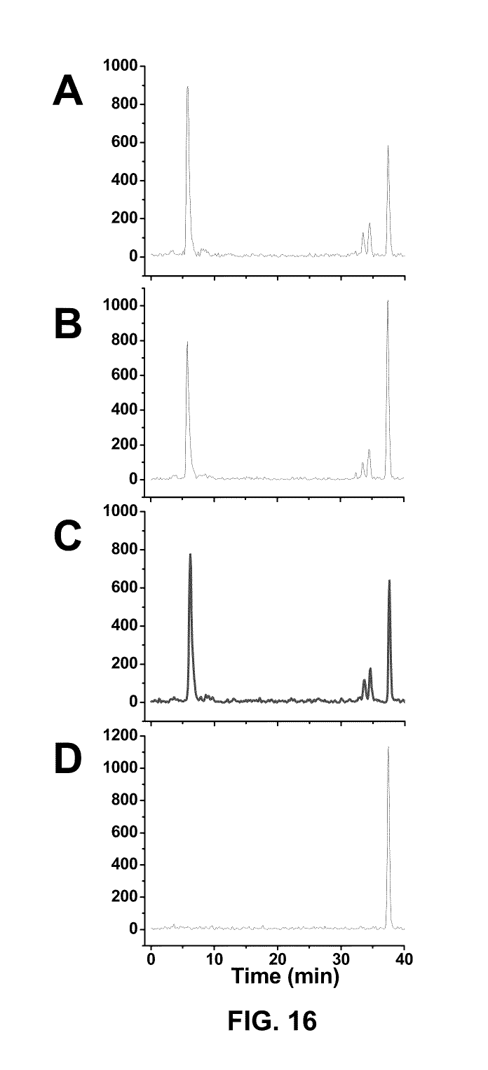

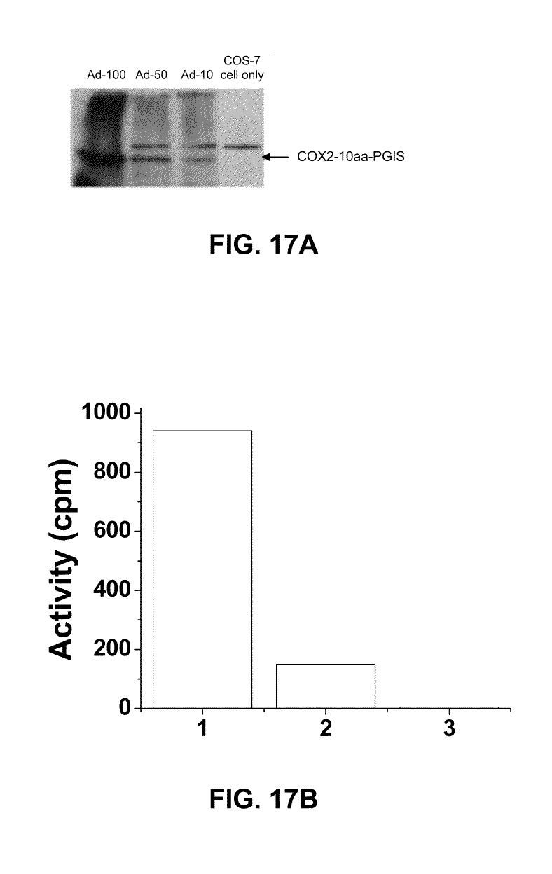

Expression of the fusion enzyme in COS-7 cells using Ad-COX2-10aa-PGIS. Expression of the fusion enzyme using the Ad-COX2-10aa-PGIS was also tested in COS-7 cells. In previous studies it was found that the human fusion enzyme shows higher expression level yield in HEK293 cells than COS-7 cells when using the pcDNA3.1 vector. To see whether the adenovirus system could improve this expression, the COS-7 cells cultured in 6-wells plate were transfected with different amounts of the Ad-COX2-10aa-PGIS. The expression yield of the fusion enzyme was dramatically increased in the COS-7 cells by the viral system compared to that of the use of pcDNA3.1 vector. These findings were further confirmed by enzyme activity assays (FIG. 16) and western blot analysis (FIG. 17A). In FIG. 16, assay results using 100 μL (A), 50 μL (B) and 10 μL (C) of the Ad-COX2-10aa-PGIS are shown. The Ad virus without the fusion enzyme gene was used as control (D).

In FIG. 17, the Western blot analysis for COX2-10aa-PGIS fusion enzyme expressed in the COS-7 cells is shown in (A). The expressed fusion enzyme in the transfected cells using 10-100 μL of the viral stock were separated by 7% PAGE and then analyzed by Western blot using anti-PGIS antibody. The expressed fusion enzyme is indicated with an arrow. In the bar graph (B), the results of the enzyme activity assay for the recombinant COX2-10aa-PGIS expressed in COS-7 cells are shown. The procedure used for these activity assays is the same as in FIG. 14B. The fusion enzyme was expressed by Ad viral system (1), pcDNA3.1 vector (2). The untransfected cells were used as negative control (3). The expression yield of the fusion enzyme in COS-7 cells using the Ad system was approximately 5-folds higher than when using a non-viral vector (FIG. 17B). Similar expression yields were obtained for the COS-7 and in HEK293 cells using the Ad system. These results further indicate that the newly constructed viral transfection system of Ad-COX2-10aa-PGIS is suitable for gene transfection in mammalian cells.

Expression of the fusion enzyme in primary-cuftured cells using Ad-COX2-10aa-PGIS. The successful expression of the COX2-10aa-PGIS fusion enzyme with the adenovirus system allowed this work to move forward to testing for the introduction of the fusion enzyme cDNA into primary-cultured cells, which are models for the gene transfection and gene therapy in vivo. Stromal cells isolated from mouse or rabbit adipose tissue were selected to test the transfection using the viral system.

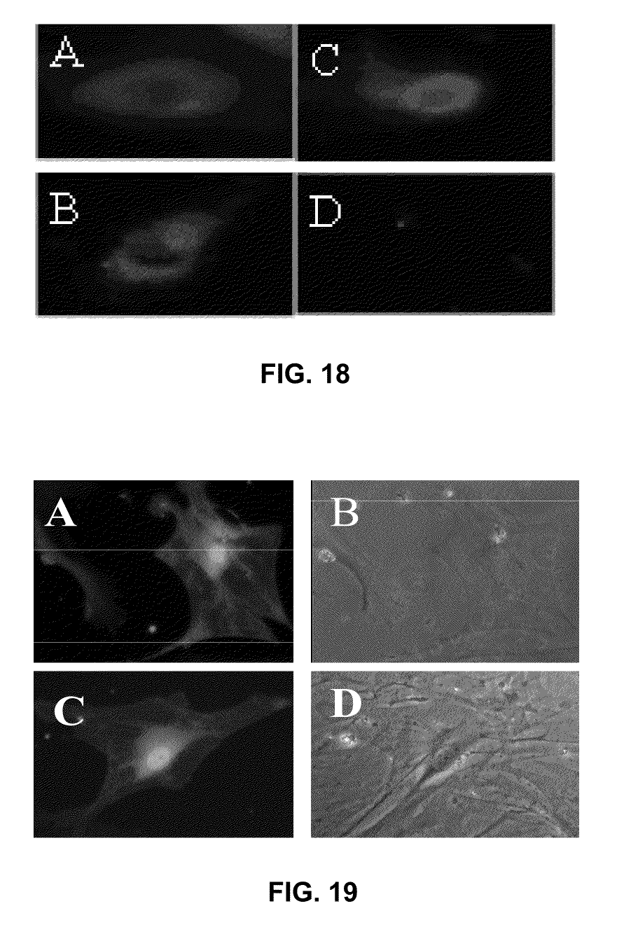

Isolation and culture of the stromal cells isolated from adipose tissue. It has been reported that CD34 positive stromal-vascular fraction (SVF) cells isolated from adipose tissue were able to differentiate into endothelial cells in vitro and in vivo. These cells have the stem cell properties of reproducibility and further differentiations. Based on the reported procedures, the CD34 positive SVF cells were isolated from mouse or rabbit adipose tissues and cultured as a source for the gene transfer. To see whether the primary-cultured cells could take in the cDNA of the fusion enzyme, the cells are transfected by the Lipofectamine method with a pcDNA3.1 vector containing the cDNA of the fusion enzyme linked to a red fluorescence protein (RFP) (COX2-10aa-PGIS-RFP). The expression of the fusion enzyme with the RFP tag in the SVF cells was observed (FIG. 18). Fluorescence micrographs of CD34-positive SVF (A) and an ECL cell (B) differentiated from an adipocyte, demonstrating transient over-expression of COX-2-10aa-PGIS with an RFP tag. Whereas (C) is an SFV cell line with a stable expression of the COX-2-10aa-PGIS with the RFP tag and (D) is a non-transected SVF cell used as negative control (D). This experiment indicated that the newly engineered COX2-10aa-PGIS could be overexpressed in the primary-cultured SVF cells, which is suitable for gene transfer and gene therapy in vivo.

Transfection of SVF cells using the adenovirus containing green fluorescence protein (GFP) cDNA. As described above in the instant example, expression of the active COX2-10aa-PGIS in HEK293 and COS-7 cells could be achieved by viral (adenovirus) as well as the non-viral vector (pcDNA3.1). However, the expression yield of the fusion enzyme is 6-7-folds higher using viral vector than that of non-viral vector. Theoretically higher expression of the fusion enzyme in the transfected cells will produce more PGI2. To test if the SVF cells, isolated and derived from the adipose tissue, can be transfected by viral gene delivery systems, the GFP cDNA (subcloned into an Ad vector) was used to transfect the cells. In FIG. 19, ECL cells (A, B) and SVF (C,D) cells transfected with adenovirus containing GFP cDNA. (A) and (C) are green fluorescent pictures. (B) and (D) are regular light pictures. FIG. 19 shows the over-expressed GFP in the ECL cells and SVF cells, which indicates that the adenovirus can be used as a viral delivery system for the primary-cultured SVF cells.

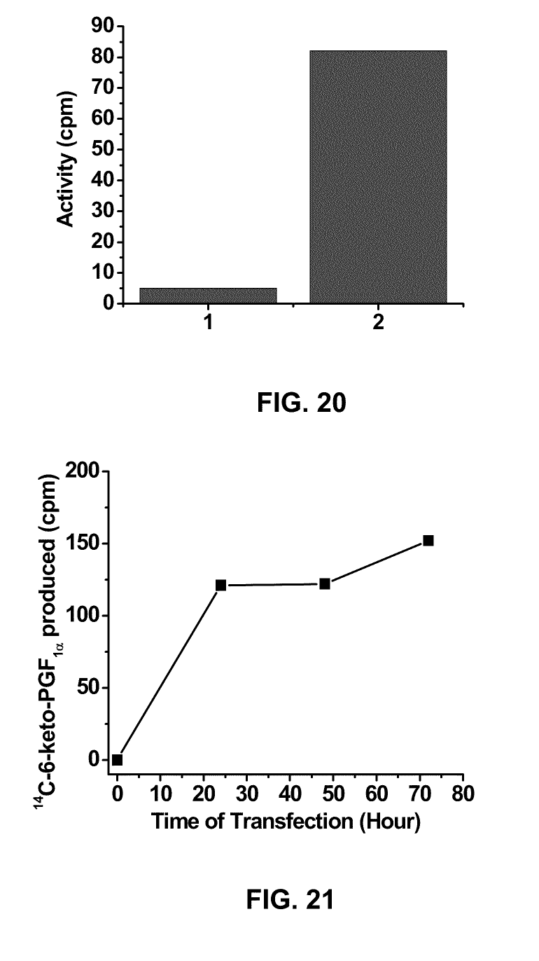

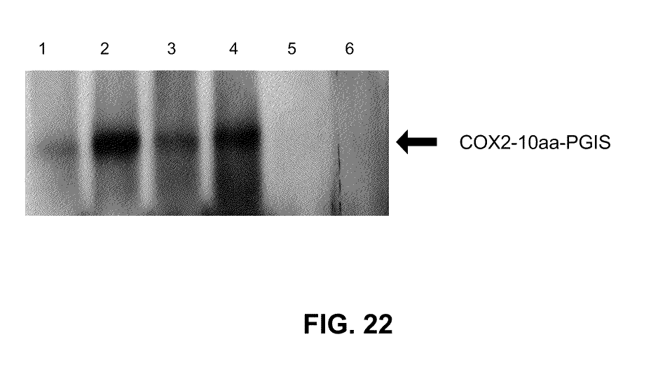

Transfection of the primary-cultured SVF cells using Ad-COX2-10aa-PGIS. The results described above have confirmed that the newly engineered COX2-10aa-PGIS could be overexpressed in the primary-cultured stromal cells and that the adenovirus-mediated gene transfer is suitable for gene delivery into the cells. It was then demonstrated that the COX2-10aa-PGIS could overexpress in the stromal cells with activity to specifically up-regulate the biosynthesis of the vascular protector, PGI2. The conditions used for the gene transfection of the fluorescence protein tagged COX2-10aa-PGIS in SVF cells proved to be the optimum conditions for transfection of the primary culture cells using the viral vector containing the cDNA of the fusion enzyme cDNA. To exclude the possibility of contamination of the vascular cells (which can endogenously produce PGI2) in the SVF cells, the background of the PGI2 production in the SVF cells was determined. After addition of [14C]-AA to the control adenovirus -transfected SVF cells (without the fusion enzyme gene), little PGI2 production was observed (bar (1) in FIG. 20). Non-endogenous PGI2 production in the stromal cells indicated that non-significant contamination of vascular cells are in the prepared SVF cells. Transfection of the cells using Ad-COX2-10aa-PGIS revealed that the PGI2 biosynthesis was 20-folds higher in comparison to the background (bar (2) in FIG. 20). A time course study was performed to investigate the expression of the fusion enzyme in the SVF cells using the viral vector (FIG. 21). After 24-hours of the viral transfection, the enzyme expression has reached 85% confluence. However, the expression could be extended to 72-hours with a noticeable increase in the enzyme activity (FIG. 21). Western blot analysis further confirmed the increasing expression of the fusion enzyme in the SFV cells by the viral transfection system (FIG. 22). The fusion enzyme (CPX2-10aa-PGIS) expression using the viral vector corresponds to lanes 1-4) and lane 6 corresponds to the fusion enzyme expression using the non-viral vector. The rabbit SVF cells transfected with 100 μL (for 2 (lane 1) or 3 (lane 2) days), or 50 μl (for (2 (lane 3) or 3 (lane 4) days) of the viral system of Ad-COX2-10aa-PGIS, or non-viral vector of pcDNA3. 1 -COX2-10aa-PGIS for 3 days were analyzed by Western blot as described with respect to FIG. 15). The non-transfected SVF cells were used as negative control (lane 5). The expression yield using the viral system is much higher (FIG. 22, lanes 1-4) than that of the non-viral pcDNA3.1 system (FIG. 22, lane 6). These results indicated that the Ad-COX2-10aa-PGIS could be used to deliver the cDNA of the newly engineered COX2-10aa-PGIS to the stromal cells for up-regulation of PGI2 biosynthesis, which carries the potential for protection of the ischemic diseases including stroke, heart disease and vascular damages of diabetes in vivo.

### Example 8

Large-scale Expression of Newly Engineered COX2-10aa-PGIS Using Baculovirus System.

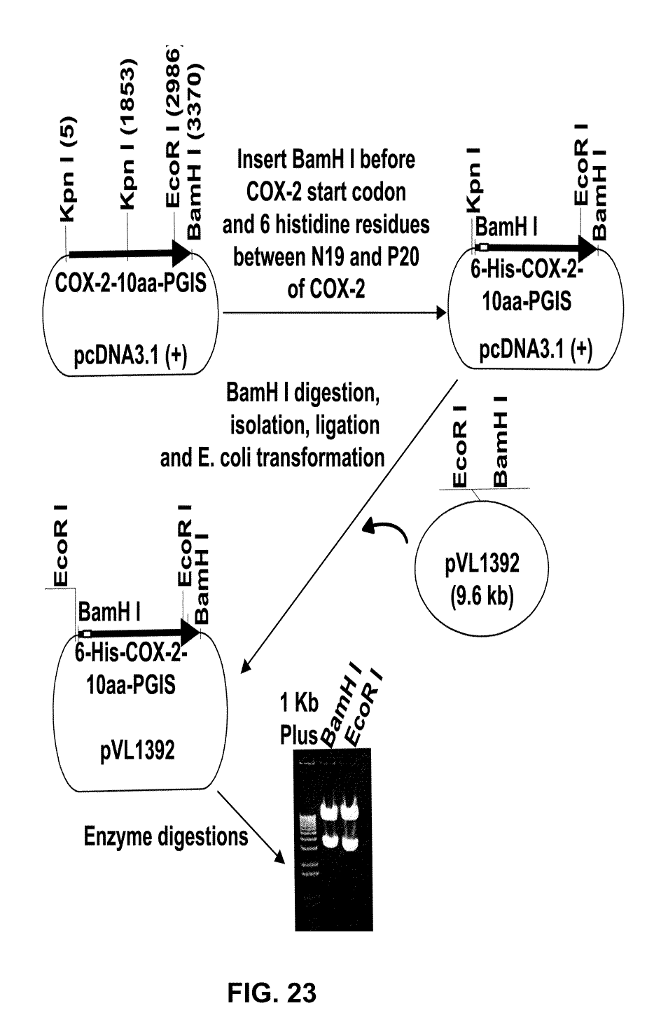

Subcloning of the Active COX2-10aa-PGIS cDNA into Baculovirus (BV) vector. To test whether a small amount of fusion enzyme expressed in the HEK293, COS-7 and stromal cells can be converted to a large-scale protein expression and preparation, a BV expression system widely used for preparation of large-scale recombinant protein was adopted for further studies. The steps of the gene cloning of the cDNA of the engineered humanized fusion enzyme (COX2-10aa-PGIS) into BV vector with a His-tag (which is suitable for affinity purification using a Ni-column) are shown in FIG. 23.

Pilot-expression of the COX2-10aa-PGIS Fusion Enzyme in BV system. The expression of the COX2-10aa-PGIS in BV system using the construct described above was performed in three steps:

COX2-10aa-PGIS cDNA was cloned into an appropriate BV transfer plasmid (pVL1392). Then, Autographa Californica nuclear polyhedrosis (AcNPV) was used to cotransfect the Sf9 insect cells with the pVL1392 recombinant vector containing COX2-10aa-PGIS (BV-COX2-10aa-PGIS) to get recombinant virus expression in the Sf9 cells;

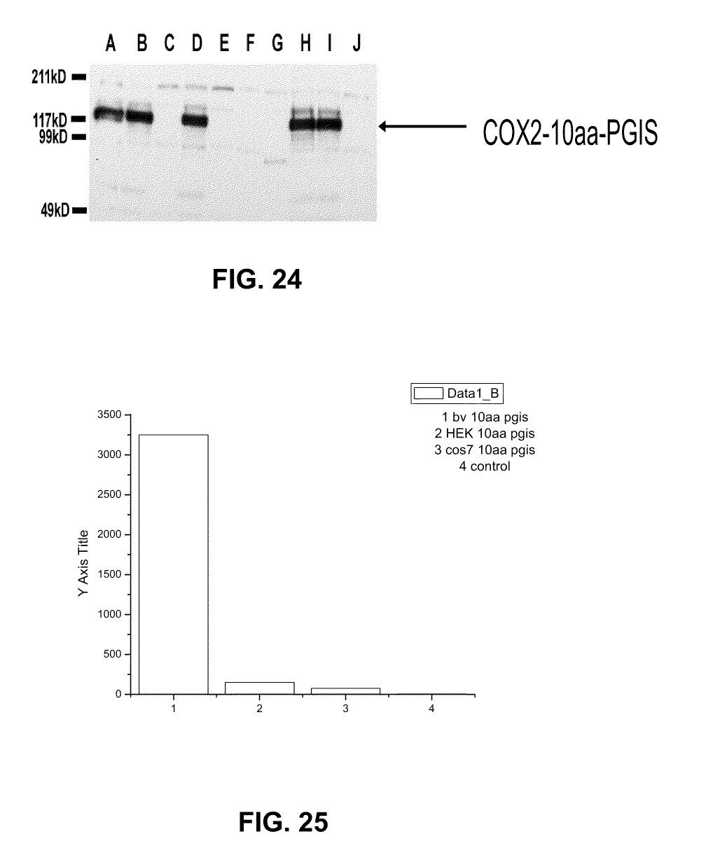

(b.) Individual clones of the BV-COX2-10aa-PGIS plaque are purified by an agarose overlay assay. The resultant viral plaques are used to infect Sf9 cells which are then screened for the expression of the fusion enzyme. High expression of the COX2-10aa-PGIS fusion enzyme in the cells was observed in five of the ten clones of the BV preparation (FIG. 24). The enzyme activities of the BV-expressed fusion enzyme were evaluated by addition of AA as an initial substrate. The BV-expressed fusion enzyme shows significant activity in converting [14C]-AA to [14C]-PGI2 (degraded into [14C]-6-keto-PGF1α (FIG. 25, lane 1). By comparison of the specific protein amounts of the fusion enzyme related to the total protein using activity assay, the expression level of the BV system is approximately 30-50-folds higher than that of expression of the fusion enzyme in COS-7 and HEK293 cells using pcDNA3.1 vector, respectively (FIG. 25).

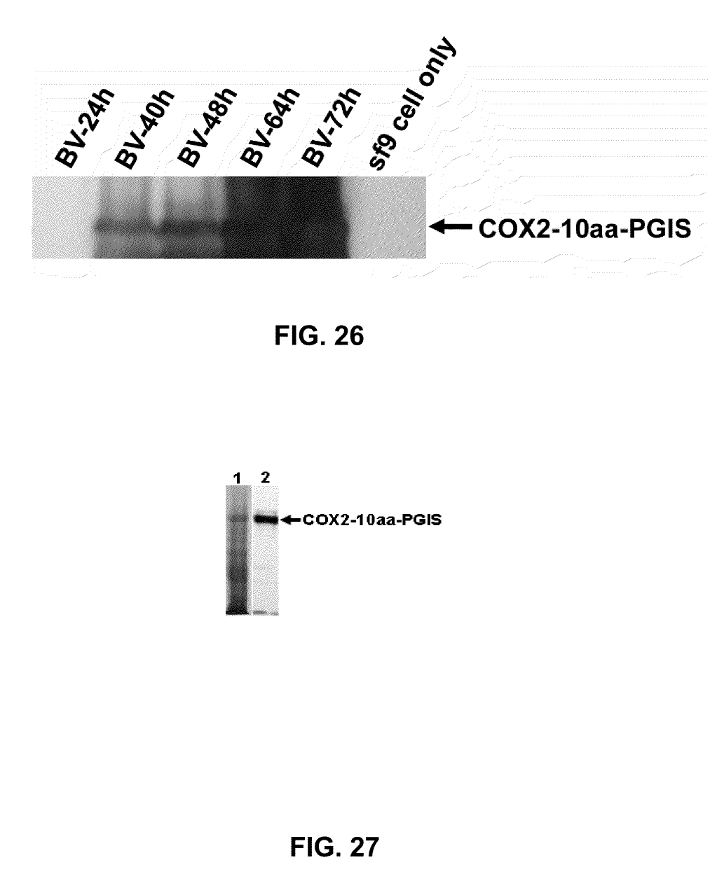

Time Course and Stability of the COX2-10aa-PGIS Fusion Enzyme Expressed in BV system. To optimize the BV expression system for COX2-10aa-PGIS, the level of the fusion enzyme expressions was observed at 24, 48, and 72 hours. An increase in expression of the fusion enzyme was observed up to 72-hours (FIG. 26). In addition, degradation of the fusion enzyme was very minimal up to the 72 hour mark as was determined by Western blot analysis using anti-COX2 and PGIS antibodies (FIG. 26). This suggests that the membrane-bound fusion enzyme is relatively stable up to 72-hours, and 72-hours expression yields more protein than that of a shorter expression time.

High Yield and Large-scale Expression of the Active Fusion Enzyme Using the BV system. It is very important to have a high yield and large-scale expression system to prepare a sufficient amount of the enzyme protein for detailed analyses and tests for protein therapy in ischemic tissues. The best BV clone expressing COX2-10aa-PGIS in the pilot-expression studies (FIG. 24) was selected for high yield and large-scale expression. The expression of the COX2-10aa-PGIS was performed in a 500 ml-culture of the BV transfected Sf9 cells. After 72 hours, the cells were harvested and a total of 320 mg protein was obtained. The expression yield of the specific COX2-10aa-PGIS was estimated by Coomassie Blue staining analysis. The band for this enzyme showed up in the position with a molecular weight of 135 kDa in the Coomassie Blue staining was further identified by the corresponding Western blot (FIG. 27). 5% specific amount of the fusion enzyme in the total protein was estimated from the Coomassie Blue staining by density scanning analysis. 500 ml of the BV-transfected Sf9 cells contained approximately 16 mg of the fusion enzyme, which is enough for most spectroscopic analyses, and tests of the protein therapy for ischemic tissue in mouse model.

Comparison of the Triple catalytic functions of the membrane-bound fusion enzyme expressed in the BVsystem and in HEK293/COS-7 cells. In many cases, large-scale expression of the recombinant protein in insect cell systems, such as the BV system could have mis-folding leading to the production of mis-folded protein without biological activities. To test whether the BV-expressed fusion enzyme retained its full activity like that of the fusion enzyme expressed in HEK293, COS-7, and stromal cells, the membrane-bound fusion enzyme prepared from the BV expression system was assayed and its specific triple-catalytic functions were compared with that of the active fusion enzyme expressed in HEK293 and COS-7 cells which used the pcDNA3.1 vector. When the enzyme activities were calibrated by the amounts of the specific fusion enzyme proteins expressed in BV system and pcDNA3.1, the specific enzyme activities for converting AA to PGI2 are identical. This observation suggested that the fusion enzyme expressed by the BV system has similar folding and membrane anchor topology as that of the HEK293 and COS-7 cell expression system with the pcDNA 3.1 vector, and is suitable for further enzyme property evaluation and protein therapy.

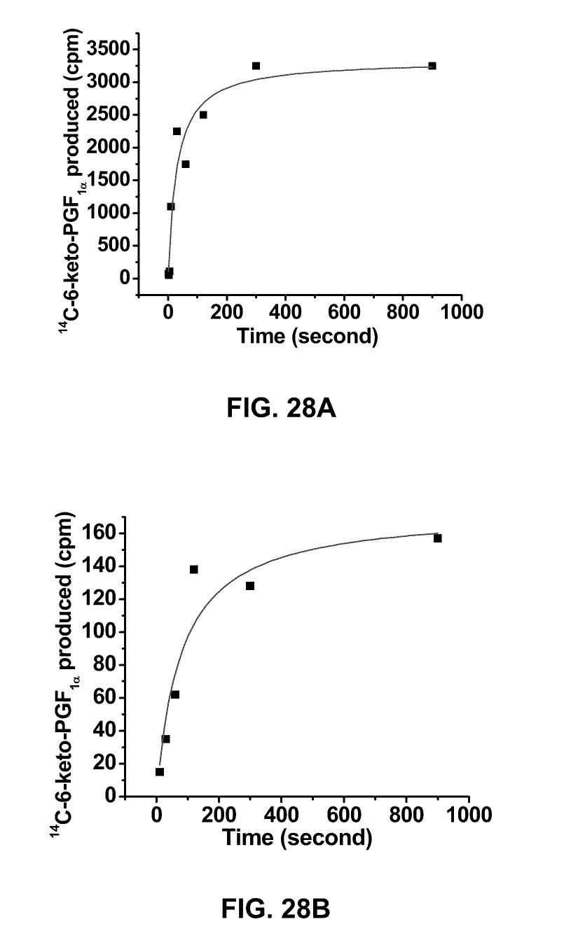

Superiority of the fusion enzyme over the co-expressed COX2 and PGIS in terms of the enzyme initial velocities. The large-scale expression of the fusion enzyme by the BV system can produce the necessary amount of protein for further characterization of the enzyme properties. The initial velocity of the membrane bound fusion enzyme was compared with the co-expressed individual COX2 and PGIS. Previous studies have indicated that the substrate presentation from COX to PGIS occurs through a “channel” formed by N-terminal membrane anchor domain and the F/G loop anchored to the ER membrane of PGIS. Thus the PGH2 produced from COX may accumulate in the hydrophobic channel and then present to the active site of PGIS. Based on this theory the fusion enzyme with a transmembrane (TM) domain linked to COX2 and PGIS in the ER membrane is an ideal connection with minimum alteration to the “channel” for PGH2 movement from COX to PGIS. In addition, the TM domain shall further stabilize the “channel” structure and produce a more hydrophobic environment in the “channel” to help with the accumulation of the lipid molecule, PGH2. This will also shorten the coupling reaction between the catalytic functions of COX and PGIS. A time course study was used to test this hypothesis. As shown in FIG. 28, the fusion enzyme displayed a much faster initial reaction kinetic compared to the mixture of the individual COX2 and PGIS anchored in the ER membrane. To reach the 50% of the maximum activity, the BV-expressed COX2-10aa-PGIS needed only 25 seconds (FIG. 28A), but the co-expressed individual COX2 and PGIS required 75 seconds (FIG. 28B). This indicated that the fusion enzyme is 3-fold faster to convert AA to PGI2 than that of the mixture of the individual enzymes. The fusion enzyme demonstrated superiority for the initial reaction in comparison to the mixture of the individual enzymes also further supports the structural studies used for establishing the “channel” theory.

Km values of the membrane-bound fusion enzyme compared to the mixture of the individual COX2 and PGIS. The Km value for COX2 and PGIS have been previously characterized by several groups. Table 1 summarizes the studies for the Km values.

TABLE 1
Comparison of the Km values of COX2-10aa-PGIS
and the individual enzymes

| | COX2-10aa-PGIS | COX2 | PGIS |
|---|---|---|---|
| Km | 3.2 μM (expressed in HEK cells) | 6.5 μM¹ | 13.3 μM² |
| | 4 μM (expressed by BV system) | 0.9 μM³ | 30 μM⁴ |
| | | | 9.5 μM⁵ |

For the fusion enzyme there are three catalytic functions, its Km value represents one of the limiting catalytic activities, which most likely belongs to PGIS, because COX2 has lower Km values than that of PGIS. The Km of the COX2-10aa-PGIS expressed in HEK293 and COS-7 cells is approximately 3.2 μM. Similar Km values were observed for the fusion enzyme expressed by BV system (Table 1), which showed that Km value of the fusion enzyme for directly converting AA to PGI2 was significantly less than that of the individual PGIS, indicating that the fusion enzyme has an advantage over the mixture of the COX2 and PGIS for PGI2 biosynthesis in vivo.

In present day gene therapy, introducing the COX gene alone into cells may not increase the biosynthesis of PGI₂ due to the limited amount of endogenous PGIS in the cells. Introducing the PGIS gene alone into the cell may also not increase PGI₂ synthesis due to the limited amount of endogenous COXs in the cells. Co-introducing un-linked COX and PGIS genes into cells is reasonably anticipated to increase the difficulty of gene delivery and would not provide specific control of PGI₂ production due to the competition of the other downstream synthases, which share PGH₂ as their substrate. For example, it is particularly difficult to avoid the over-producing of PGE₂ when large amount of the microsomal PGES-1 is induced by the tissue injuries during the gene delivery. The presently engineered single COX-2-linker-PGIS eliminates the step of PGH₂ movement from the COX protein to the PGIS protein, which are normally in separate locations, leading to increased PGI₂ production and limiting PGH₂ availability for TXA₂ and PGE₂ production. Thus, the hybrid molecules and compositions offer a new generation of cDNA for COX gene therapy.

Alternatively, the COX-linker-PGIS protein may be used as a therapeutic reagent to instantly increase PGI₂ production locally through injection of the engineered protein. It is particularly interesting that COX-2 inhibitors inhibit COX-2 activity but not the COX-1 activity. Thus, introduction of the COX-1-linker-PGIS to vascular systems may be used to overcome the damage of the vascular functions caused by COX-2 inhibitors. Given the importance of PGI₂ in vascular diseases and thrombosis, the presently engineered COX-linker-PGIS will be a useful therapeutic molecule in vivo.

While the present invention has been described in reference to illustrative embodiments, this description is not intended to be construed in a limiting sense. Various modifications and combinations of the illustrative embodiments, as well as other embodiments of the invention, will be apparent to persons skilled in the art upon reference to the description. For example, in the foregoing examples, certain 10aa and 22aa linker peptides are employed. The results obtained with those linker sequences are considered representative of other peptides with different sequences and/or intermediate sequence lengths between 10aa and 22aa that may also serve as satisfactory linker sequences. Such other satisfactory sequences, in a similar hybrid protein, must be able to span the cell membrane and permit the COX and PGIS to assume membrane topographies similar to their native counterpart enzymes, to fold correctly, and to demonstrate enzymatic activity similar to that of the native COX and PGIS. Further, this disclosure provides evidence that the COX enzymes can be linked with other downstream eicosanoid-synthesizing (ES) enzymes, thereby generating other linked proteins, to specifically regulate the biosynthesis of eicosanoids, which play diverse and potent biological functions in human. Thus, PGIS is considered to be representative of other eicosanoid-synthesizing enzymes that may be used to construct similarly successful hybrid proteins. It is therefore intended that the appended claims encompass any such modification or embodiments. Each and every original claim is incorporated into the specification as an embodiment of the present invention. Thus the original claims are a further description and are an addition to the preferred embodiments of the present invention. The disclosures of all patents, patent applications and publications cited herein are hereby incorporated herein by reference, to the extent that they provide exemplary, procedural or other details supplementary to those set forth herein.

### REFERENCES CITED

1. Kobayashi T, Narumiya S. (2002) Function of prostanoid receptors: studies on knockout mice. Prostaglandins Other Lipid Mediat. 68-69, 557-573.
2. Narumiya, S. and FitzGerald, G. A. (2001) Genetic and pharmacological analysis of prostanoid receptor function. J. Clin. Invest. 108, 25-30.
3. Sugimoto, Y., Narumiya, S. and Ichikawa A. (2000) Distribution and function of prostanoid receptors: studies from knockout mice. Prog. Lipid Res. 39, 289-314.
4. Kaliman, J., Sinzinger, H., Staudacher, M. and Mannheimer, E. (1985) Defects in the prostaglandin system. II. Familial platelet-prostacyclin receptor defect (Wien-Hietzing defect)—pathogenetic significance for (early) development of atherosclerosis? Wien Klin Wochenschr 97, 323-326).
5. Issemann, I. and Green, S. (1990) Activation of a member of the steroid hormone receptor superfamily by peroxisome proliferators. Nature 347, 645-650.
6. Zhang, J., Fu, M., Zhu, X., Xiao, Y., Mou, Y., Zheng, H., Akinbami, M. A., Wang, Q. and Chen, Y. E. (2002) Peroxisome proliferator-activated receptor delta is up-regulated during vascular lesion formation and promotes post-confluent cell proliferation in vascular smooth muscle cells. J. Biol. Chem. 277, 11505-11512.
7. Plutzky, J. (2003) Medicine. PPARs as therapeutic targets: reverse cardiology? Science 302, 406-407).
8. Arbab, F., Goldsby, J., Matijevic-Aleksic, N., Huang, G., Ruan, K. H. and Huang, J. C. (2002) Prostacyclin is an autocrine regulator in the contraction of oviductal smooth muscle. Hum. Reprod. 17, 3053-3059.
9. Huang, J. C., Arbab, F., Tumbusch, K. J., Goldsby, J. S., Matijevic-Aleksic, N. and Wu, K. K. (2002) Human fallopian tubes express prostacyclin (PGI) synthase and cyclooxygenases and synthesize abundant PGI. J. Clin. Endocrinol. Metab. 87, 4361-4368.
10. Majerus, P. W. (1983) Arachdonate metabolism in vascular disorders. J. Clin. Invest. 72, 1521-1525.
11. Pace-Asciak, C. R. and Smith, W. L. (1983) Enzymes in the biosynthesis and catabolism of the eicosanoids: prostaglandins, thromboxanes, leukotrienes and hydroxy fatty acids. Enzymes 16, 544-604.
12. Samuelson, B., Goldyne, M., Granstrom, E., Hamberg, M., Hammarstrom, S. and Malmsten, C. (1978) Prostaglandins and thromboxanes. Ann. Rev. Biochem. 47, 994-1030.
13. Picot, D., Loll, P. J. and Garavitok, R. M. (1994) The X-ray crystal structure of the membrane protein prostaglandin H2 synthase-1. Nature 367, 243-249.
14. Luong, C., Miller, A., Barnett, J., Chow, J., Ramesha, C., Browner, M. F. (1996) Flexibility of the NSAID binding site in the structure of human cyclooxygenase-2. Nat. Struct. Biol. 3, 927-933.
15. Lin, Y. Z., Deng, H. and Ruan, K.-H. (2000) Topology of catalytic portion of prostaglandin 1(2) synthase: identification by molecular modeling-guided site-specific antibodies. Arch. Biochem. Biophys. 379, 188-197.
16. Deng, H., Huang, A., So, S.-P., Lin, Y.-Z. and Ruan, K-H. (2002) Substrate access channel topology in membrane-bound prostacyclin synthase. Biochem J. 362, 545-551.
17. Deng, H., Wu, J., So, S.-P. and Ruan, K.-H. (2003) Identification of the residues in the helix F/G loop important to catalytic function of membrane-bound prostacyclin synthase. Biochemistry 42, 5609-5617.
18. Ruan, K. H. (2004) Advance in understanding the biosynthesis of prostacyclin and thromboxane A₂ in the endoplasmic reticulum membrane via the cyclooxygenase pathway. Mini Rev. Med. Chem. 4, 639-647.
19. Ruan, K.-H., Deng, H., So, S.-P., and Jiaxin Wu. The N-terminal membrane domain of the membrane-bound prostacyclin synthase involved in the substrate presentation in the coupling reaction with cyclooxygenase. Arch. Biochem. Biophys, 2005 (in press).
20. Jiang H, Peterson R S, Wang W, Bartnik E, Knudson C B, Knudson W. (2002) A requirement for the CD44 cytoplasmic domain for hyaluronan binding, pericellular matrix assembly, and receptor-mediated endocytosis in COS-7 cells. J Biol Chem. 22, 277(12):10531-8.
21. Ren, Y., Walker, C., Loose-Mitchell, D. S., Deng, J., Ruan, K.-H. and Kulmacz, R. J. (1995) Topology of prostaglandin H synthase-1 in the endoplasmic reticulum membrane. Arch. Biochem. Biophys. 323, 205-214.

1. An isolated hybrid protein molecule comprising a cyclooxygenase (COX) amino acid sequence and an eicosanoid-synthesizing (ES) enzyme amino acid sequence with a linker sequence disposed therebetween and directly connecting said COX enzyme sequence to said ES enzyme sequence.

2. The hybrid protein molecule of claim 1 wherein said linker sequence is about 10 to 22 amino acids long.

3. The hybrid protein molecule of claim 2 wherein said linker sequence is His-Ala-Ile-Met-Gly-Val-Ala-Phe-Thr-Trp (SEQ ID NO. 1) or His-Ala-Ile-Met-Gly-Val-Ala-Phe-Thr-Trp-Val-Met-Ala-Leu-Ala-Cys-Ala-Ala-Pro-Pro-Leu-Val (SEQ ID NO. 2) or residues 1-11, 1-12, 1-13, 1-14, 1-15, 1-16, 1-17, 1-18, 1-19, 1-20 or 1-21 of SEQ ID NO. 2.

4. The hybrid protein of claim 1, wherein said linker peptide provides approximately 10 Å separation between the catalytic sites of said COX and said ES enzyme, said connected enzymes being capable of substantially normal folding and enzymatic activity compared to the native folding and enzymatic activity of the native COX and ES enzymes.

5. The hybrid protein of claim 1 comprising cyclooxygenase (COX), a transmembrane linker, and a prostacyclin synthase (PGIS), wherein said hybrid protein is either chemically synthesized or recombinantly produced.

6. A pharmaceutical composition comprising:
the hybrid protein of claim 1; and
a pharmaceutically acceptable carrier.

7. An isolated DNA sequence comprising:
a sequence encoding a cyclooxygenase (COX);
a sequence encoding an eicosanoid-synthesizing enzyme (ES); and
a sequence encoding a transmembrane linker peptide directly connecting said COX and ES sequences.

8. The isolated DNA sequence of claim 7, wherein the cyclooxygenase is a cyclooxygenase isoform-1 (COX-1) or a cyclooxygenase isoform-2 (COX-2).

9. The isolated DNA sequence of claim 7, wherein the eicosanoid-synthesizing enzyme is a prostacyclin synthase (PGIS).

10. The isolated DNA sequence of claim 7 wherein the sequence encoding said transmembrane linker peptide encodes the amino acid sequence His-Ala-Ile-Met-Gly-Val-Ala-Phe-Thr-Trp (SEQ ID NO. 1) or the amino acid sequence His-Ala-Ile-Met-Gly-Val-Ala-Phe-Thr-Trp-Val-Met-Ala-Leu-Ala-Cys-Ala-Ala-Pro-Pro-Leu-Val (SEQ ID NO. 2) or the sequence corresponding to residues 1-11, 1-12, 1-13, 1-14, 1-15, 1-16, 1-17, 1-18, 1-19, 1-20 or 1-21 of SEQ ID NO. 2.

11. A virus vector comprising the DNA sequence according to claim 7.

12. The vector of claim 11, wherein said virus is adeno-associated virus.

13. A host cell containing an expressible DNA sequence comprising the isolated DNA sequence of claim 7.

14. A process for producing a hybrid protein comprising a cyclooxygenase (COX), a transmembrane linker, and a prostacyclin synthase (PGIS), the process comprising:
culturing the host cell of claim 13 under conditions suitable for expression of the DNA encoding said hybrid protein molecule, and
recovering a biologically active hybrid protein molecule comprising a cyclooxygenase (COX), a transmembrane linker, and a prostacyclin synthase (PGIS).

15. A method of treating an individual having a vascular disease, or at risk of developing a vascular disease, said method comprising microinjecting an effective amount of the pharmaceutical composition of claim 6 into at least one vascular cell in said individual, to cause the production of at least one biologically active compound that deters or prevents the occurrence of said vascular disease in said individual, or ameliorates existing vascular disease.

16. The method of claim 15 wherein one said biologically active compound is PGI₂.

17. The method of claim 15 wherein said vascular disease comprises at least one condition chosen from the group consisting of stroke, heart attack, thrombosis, ischemia, and inflammation in an organ or vessel of the individual's vascular system.

18. A method of treating an individual having a vascular disease, or at risk of developing a vascular disease, said method comprising transfecting at least one vascular cell in said individual with the vector of claim or microinjecting said vector into said at least one vascular cell, to cause the production of a functional hybrid protein comprising said COX, said linker peptide and said ES enzyme, whereby at least one biologically active compound is produced which deters or prevents the occurrence of said vascular disease in said individual, or ameliorates existing vascular disease.

19. The method of claim 18 wherein said vascular disease comprises at least one condition chosen from the group consisting of stroke, heart attack, thrombosis, ischemia, and inflammation in an organ or vessel of the individual's vascular system.

20. The method of claim 18, wherein a COX-2 inhibitor is administered to said individual, and wherein said hybrid protein contains the structure COX-1-linker-PGIS, and the COX-1 enzymatic activity of said hybrid protein counteracts at least some of the adverse vascular effects arising from COX-2 inhibitor administered to said individual.
